SEQUENCE LISTING (17 sequences)

SEQ ID NO: 1 - DNA, 20 bp, Artificial Sequence
  15020 mutant reverse primer
ctgaacttgtggccgtttac

SEQ ID NO: 2 - DNA, 20 bp, Artificial Sequence
  26840 wild type reverse primer
gtctggtcagaatgcccttg

SEQ ID NO: 3 - DNA, 20 bp, Artificial Sequence
  8060 Common primer
ctgctctctgctcccagtct

SEQ ID NO: 4 - DNA, 22 bp, Artificial Sequence
  Forward primer for genotyping mice
tctgacatccgctcagcaccaa

SEQ ID NO: 5 - DNA, 21 bp, Artificial Sequence
  Reverse primer for genotyping mice
gggcccgcccaaatataaacc

SEQ ID NO: 6 - DNA, 21 bp, Artificial Sequence
  Reg3b F forward primer
actccctgaagaatataccct

SEQ ID NO: 7 - DNA, 21 bp, Artificial Sequence
  Reg3b R reverse primer
gctattgagcacagaracgag

SEQ ID NO: 8 - DNA, 22 bp, Artificial Sequence
  Reg3g F forward primer
atgcttccccgtataaccatca

SEQ ID NO: 9 - DNA, 22 bp, Artificial Sequence
  Reg3g R reverse primer
ggccatatctgcatcataccag

SEQ ID NO: 10 - DNA, 20 bp, Artificial Sequence
  Lysp F forward primer
atggctaccgtcgrgrcaag

SEQ ID NO: 11 - DNA, 20 bp, Artificial Sequence
  Lysp R reverse primer
cggtctccacggttgtagtt

SEQ ID NO: 12 - DNA, 20 bp, Artificial Sequence
  Gapdh F forward primer
gacatgccgcctggagaaac

SEQ ID NO: 13 - DNA, 19 bp, Artificial Sequence
  Gapdh R reverse primer
agcccaggatgcccttagt

SEQ ID NO: 14 - DNA, 20 bp, Artificial Sequence
  803F primer
aaactyaaakgaattgrcgg

SEQ ID NO: 15 - DNA, 15 bp, Artificial Sequence
  1392R primer
acgggcggtgwgtrc

SEQ ID NO: 16 - DNA, 51 bp, Artificial Sequence
  Adapter sequence 803F
  misc_feature (45)..(45): n is a, c, g, or t
tcgtcggcagcgtcagatgtgtataagagacagttagakacccbngtagtc

SEQ ID NO: 17 - DNA, 64 bp, Artificial Sequence
  Adapter sequence 1392wR
gtctcgtgggctcgggtctcgtgggctcggagatgtgtataagagacagacgggcggtgwgtrc

(19) United States
(12) Patent Application Publication
(10) Pub. No.: US 2022/0378878 A1
(43) Pub. Date: Dec. 1, 2022

(54) TREATMENT OF GASTROINTESTINAL DISEASE

(71) Applicant: The Council of The Queensland Institute of Medical Research, Herston, Queensland (AU)

(72) Inventors: Geoffrey R. HILL, Herston, Queensland (AU); Andrea S. HENDEN, Herston, Queensland (AU); Kate H. GARTLAN, Herston, Queensland (AU)

(73) Assignee: The Council of The Queensland Institute of Medical Research, Herston, Queensland (AU)

(21) Appl. No.: 17/774,782

(22) PCT Filed: Dec. 18, 2020
(86) PCT No.: PCT/AU2020/051389

§ 371 Date: May 5, 2022

(30) Foreign Application Priority Data
Dec. 20, 2019 (AU) 2019904873

(51) Int. Cl. A61K 38/21; A61K 38/20; A61P 1/04

(52) U.S. Cl. CPC A61K 38/21; A61K 38/20; A61P 1/04; A61K 2035/124

(57) ABSTRACT

Described herein are methods of protecting and/or promoting proliferation of intestinal epithelium by administering an Interferon (IFN) λ ligand or an IFNλ, receptor agonist to a subject in need thereof. Preferred methods utilising an IFNλ receptor agonist concern preventing or treating a gastrointestinal disease, disorder or condition, such as GVHD, inflammatory bowel disease and autoimmune and therapy-related enterocolitis.

### RELATED APPLICATIONS

This application claims the benefit AU Patent Application No 2019904873, filed 20 Dec. 2020, the entire contents of which are incorporated by reference herein.

### FIELD

THIS INVENTION relates to gastrointestinal diseases, disorders and conditions. More particularly, this invention relates to methods for the treatment and/or prevention of gastrointestinal diseases, disorders and conditions, such graft versus host disease (GVHD), inflammatory bowel disease (IBD) and autoimmune and therapy-related enteritis and/or colitis.

### BACKGROUND

Allogeneic bone marrow or stem cell transplantation (hereinafter referred to as HSCT or BMT) offers curative therapy for patients with high-risk haematological malignancies. Despite advances in transplantation techniques, including advanced supportive care, reduced intensity conditioning, alternative donor sources and evolutions in GVHD prophylaxis; the overall benefit remains limited by transplant-related mortality and morbidity, reducing the number of patients who will realise cure with an acceptable quality of life. Graft-versus-host disease (GVHD) represents a major component of this morbidity and mortality. Prevention or treatment of GVHD has traditionally focused on T cell targeted immune suppression and is often accompanied by impairment in pathogen-specific immunity and graft-versus-leukemia (GVL) effects. Therapies that prevent GVHD whilst preserving leukemia and pathogen-specific immunity remain a clinical imperative.

### SUMMARY

The present invention broadly relates to methods of protecting and/or promoting proliferation of intestinal epithelium, including intestinal stem cells by administering an Interferon (IFN) λ ligand or an IFNλ receptor agonist to a subject in need thereof. In a particular form, the invention relates to methods of preventing or treating a gastrointestinal disease, disorder or condition, such as GVHD, inflammatory bowel disease and autoimmune and therapy-related enterocolitis.

In a first aspect, the invention provides a method of preventing or treating a gastrointestinal disease, disorder or condition in a subject, said method including the step of administering to the subject a therapeutically effective amount of an IFNλ receptor agonist to thereby prevent or treat the gastrointestinal disease, disorder or condition in the subject.

In one embodiment, the gastrointestinal disease, disorder or condition is at least partly characterized by gastrointestinal epithelial damage and/or loss.

Suitably, the gastrointestinal disease, disorder or condition is selected from the group consisting of an inflammatory intestinal disease, an infection, an autoimmune disease, radiation-induced intestinal damage, graft versus host disease (GVHD), and any combination thereof. In one embodiment, inflammatory intestinal disease comprises inflammatory bowel disease, ulcerative colitis and/or Crohn's disease.

In a specific embodiment, the gastrointestinal disease, disorder or condition is or comprises GVHD, such as acute GVHD. In this regard, GVHD in the subject is suitably characterised by or comprises gastrointestinal epithelial damage and/or loss, such as small intestinal and/or colonic epithelial damage and/or loss.

In certain embodiments, the subject has or is to be administered an immunosuppressive agent.

In some embodiments, the IFNλ receptor agonist is administered to the subject prior to, simultaneously with and/or subsequent to administration of a transplant. In this regard, the transplant is suitably a hematopoietic stem cell transplant, such as a bone marrow transplant or more particularly an allogeneic bone marrow transplant.

Suitably, the IFNλ receptor agonist does not modulate and/or preserves graft versus leukemia (GVL) and/or graft versus tumour (GVT) effects of the transplant.

In a second aspect, the invention provides a method of promoting survival and/or proliferation of an intestinal cell, said method including the step of contacting the intestinal cell with an IFNλ receptor agonist under conditions to promote survival and/or proliferation of the intestinal cell.

In this regard, the present method can be performed either in vitro or in vivo in a subject.

Suitably, the intestinal cell is or comprises an intestinal stem cell. More particularly, the intestinal stem cell may be positive for or expresses one or more of Lgr5, Ascl2 and Smoc2. In other embodiments, the intestinal stem cell is positive for or expresses one or more further surface markers denoting an intestinal stem cell.

In one embodiment, the IFNλ receptor agonist is administered prior to, simultaneously with and/or subsequent to administration of a cytotoxic agent to the subject. To this end, the cytotoxic agent suitably is or comprises a chemotherapeutic agent and/or a radiotherapy.

Suitably, the subject has or is suffering from a gastrointestinal disease, disorder or condition, such as an inflammatory intestinal disease, an infection, an autoimmune disease, immunotherapy-induced intestinal damage, an immune deficiency, a haematological malignancy, chemotherapy-induced intestinal damage, radiation-induced intestinal damage, GVHD, and any combination thereof.

In a third aspect, the invention provides a method of performing a transplant in a subject in need thereof, said method including the step of administering to the subject a therapeutically effective amount of an IFNλ receptor agonist prior to, simultaneously with and/or subsequent to administration of the transplant to the subject.

In certain embodiments, the transplant is or comprises a hematopoietic stem cell transplant, and more particularly is or comprises a bone marrow transplant.

In one embodiment, the present method includes the further step of administering the transplant to the subject.

In a fourth aspect, the invention relates to an IFNλ receptor agonist for use in:

(a) preventing or treating a gastrointestinal disease, disorder or condition;

(b) promoting survival and/or proliferation of an intestinal cell; and/or

(c) performing a transplant;

in a subject.

In a fifth aspect, the invention resides in use of an IFNλ receptor agonist in the manufacture of a medicament for:

(a) preventing or treating a gastrointestinal disease, disorder or condition;

(b) promoting survival and/or proliferation of an intestinal cell; and/or

(c) performing a transplant;

in a subject.

Referring to the aforementioned aspects, the IFNλ receptor agonist is suitably selected from the group consisting of IFN-λ1 (IL-29), IFN-λ2 (IL-28A), IFN-λ3 (IL-28B), IFN-λ4, including fragments, variants and derivatives thereof and any combination thereof. More particularly, the IFNλ receptor agonist suitably is or comprises recombinant IL-29. Even more particularly, the IFNλ receptor agonist suitably is or comprises pegylated recombinant IL-29. Further embodiments may relate to derivatives that include one or more modifications to target the IFNλ receptor agonist, and more particularly IL-29, to a specific tissue or tissue type (e.g., the gastrointestinal tract).

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

The indefinite articles ‘a’ and ‘an’ are used here to refer to or encompass singular or plural elements or features and should not be taken as meaning or defining “one” or a “single” element or feature. For example, “a” cell includes one cell, one or more cells and a plurality of cells.

### BRIEF DESCRIPTION OF THE FIGURES

FIG. 1. IFNλ signaling in recipient tissue determines GVHD severity. A) Survival by Kaplan-Meier estimates for B6.wild-type (WT, n=31), B6.Ifnlr1^{-/-} (Ifnlr1^{-/-}, n=31), B6.Ifnar^{-/-} (Ifnar^{-/-}, n=12) or B6.Ifnlr1^{-/-}.Ifnar^{-/-} (Ifnlr1^{-/-}.Ifnar^{-/-}, n=10) recipient mice lethally irradiated (1000 cGy), and transplanted with BALB/c derived bone marrow (BM) and T cells. A non-GVHD control group received T cell depleted BM only (TCD Ifnlr1^{-/-}.Ifnar^{-/-}, n=10). Data combined from 5 experiments. B) Clinical GVHD scores. Ifnar^{-/-} and Ifnlr1^{-/-}.Ifnar^{-/-} not shown given lack of survival beyond 7 days. C) Representative images of colon and small intestine (SI) at D7 after BMT. D) Semi-quantitative GVHD histopathology scores at D7 after BMT (WT and Ifnlr1^{-/-}, n=9; TCD, n=6, combined from 2 experiments). E) Serum FITC Dextran at day 7 post-BMT (WT, n=10; Ifnlr1^{-/-}, n=9, combined from 2 experiments). F) Serum IFNγ, IL-6, TNF and IL-17A at day 4 post-BMT (WT & Ifnlr1^{-/-}, n=23; TCD, n=10, combined from 3 experiments). G) IL-28A/B measurement in naïve and irradiated (1000 cGy) sera (n=9, combined from 2 experiments) and SI and colon measurement and corrected per gram of tissue, measured in supernatant from mucosal homogenates (n=9, combined from 2 experiments). H) IL-28A/B measurement from serum and SI at days 1, 3 and 7 after lethal irradiation (1000 cGy) and transplantation with BALB/c BM and T cells or TCD only (n=9, combined from 2 experiments). I) B6D2F1 recipients were transplanted with BM and T cells from WT or Ifnlr1^{-/-} donors. Survival and J) GVHD clinical scores (GVHD groups n=12; TCD n=8; combined from 2 experiments). Data are presented as mean±SEM. P values calculated using two tailed Mann-Whitney T test. Kaplan-Meier Survival compared by Log-rank Mantel-Cox test. *p<0.05, **p<0.01, ***p<0.001, ****p<0.0001.

FIG. 2. Enhanced GVHD in Ifnlr1^{-/-} recipients is dependent on signaling in both hematopoietic and non-hematopoietic cells. A) Transplant schema for creation of BM chimeras and secondary transplants. B) Representative images of colon at D7 post-BMT. C) Semi-quantitative colon GVHD histopathology scores at D7 post-BMT (n=11 for WT→WT and WT→Ifnlr1^{-/-}, n=15 for Ifnlr1^{-/-}→WT and n=14 for Ifnlr1^{-/-}→Ifnlr1^{-/-}, combined from 2 experiments). D) Serum IFNγ and IL-6 at day 4 post-BMT. E) qPCR enumeration of Ifnlr1 transcripts from naïve WT homogenized tissue normalized to expression in liver (n=3). Data are presented as mean±SEM. P values calculated using ANOVA and Tukey' s multiple comparison. *p<0.05, **p<0.01, ***p<0.001, ****p<0.0001.

FIG. 3. Ifnlr1-signaling in recipient NK cells is responsible for the protection from GVHD mediated by hematopoietic cells. Donor BALB/c^{Luc} T cell expansion in WT and Ifnlr1^{-/-} recipients determined by bioluminescence 7 days post-BMT. A) Representative images and B)

Quantification (n=9 per group, combined from 3 experiments). C) Quantification of donor T cells in spleen at day 4 post-BMT (n=8 per group, combined from 2 experiments). D) T cell expansion in colon at day 4 post-BMT (n=6, combined from 2 experiments). E) Proportion of donor T cells at day 4 post-BMT that are donor vs. host (n=8, combined from 2 experiments). F-G) Functional capacity of splenic DCs from naïve B6 WT or Ifnlr1^{-/-} mice to stimulate BALB/c F) CD4⁺ or G) CD8⁺ T cells in a mixed lymphocyte reaction (n=3, Data from is from 1 of 2 representative experiments. H-I) BALB/c recipients were transplanted with WT or Ifnlr1^{-/-} BM+T cells at day 0 and B6.Tea^{Luc} T cells (reactive to BALB/c I-E^{d} derived TEa peptide expressed in donor B6 I-A^{b}) transferred at day 12 to assess donor-derived APC function in the GI tract as determined by bioluminescence of antigen-specific TEa T cells. H) Representative bioluminescence and I) Quantification (n=10, combined from 2 experiments). J) Proportions of donor T cells producing IFNγ in spleen at day 4 post-BMT (n=8, combined from 2 experiments). K) Dividing capacity of splenic BALB/c T cells transplanted into WT or Ifnlr1^{-/-} recipients calculated at day 4 by CFSE dilution (n=14, combined from 3 experiments). L) Proportion of annexinV⁺7AAD⁻ apoptotic donor T cells at day 4 as per K) (n=7, combined from 2 experiments). M) Number of NKp46+ cells in naïve recipient mice (WT, n=3; Ifnlr1^{-/-}, n=6). N) B6 WT or Ifnlr1^{-/-} recipients received αNK1.1 or IgG Isotype and were transplanted with BALB/c BM+T cells. IFNγ was determined in sera at day 4 post-BMT (n=9 per group, combined from 2 experiments). O-P) B6 CD45.2⁺ WT or Ifnlr1^{-/-} recipients received αNK1.1 or IgG Isotype and were transplanted with CD45.1⁺ allogeneic BALB/c BM and CD45.1⁺CD45.2⁺ syngeneic B6 BM. O) Representative FACS plots from NK-depleted recipients showing syngeneic vs allogeneic cells in spleen 48 hours post BMT. P) Index of cytotoxicity as described in Methods (n=8, combined from 2 experiments). Q) Index of cytotoxicity in spleen of WT and Ifnlr1^{-/-} recipient mice in addition to NKp46^{Cre}.Ifnlr1^{fl.fl} negative and positive recipients (n=12, combined from 2 experiments). Data are presented as mean±SEM. P values calculated using two tailed Mann-Whitney T test. *p<0.05, **p<0.01, ***p<0.001, ****p<0.0001.

FIG. 4. Ifnlr1-signaling in Lgr5⁺ intestinal stem cells is responsible for the protection from GVHD mediated by non-hematopoietic cells. A) Heat map displaying differentially abundant OTUs consistently increased or decreased in the mice separately housed or co-housed B6 WT and/or Ifnlr1^{-/-} mice. Co-housing was performed for 4 weeks prior to transplantation (n=10, combined from 2 experiments). B) Principal Component Analysis (PCA) of fecal microbial composition for mice as in A). C) Survival of recipients in A) transplanted with BALB/c BM+T cells D) Enumeration and E) Representative images of Paneth cells from small intestine at day 7 post-transplant (WT, n=8, Ifnlr1^{-/-}, n=7, TCD, n=6 combined from 2 experiments). F) qPCR enumeration of Cryptidins (pan-cryptidin), Lysozyme P (Lysp) and Regenerating islet-derived protein III gamma (Reg3g) defensin expression from WT and Ifnlr1^{-/-} recipient mice lethally irradiated (1000 cGy), and transplanted with BALB/c derived bone marrow (BM) and T cells (n=4). G) Representative images, H) Numbers and I) Size of GI organoids grown from colonic crypt isolates and enumerated at D5 (n=6-7, combined from 3 experiments). J) Representative images and K) Semi-quantitative GVHD histopathology scores at D7 after BMT from tamoxifen treated Cre positive or negative Lgr5^{Cre}.Ifnlr1^{fl.fl} recipient mice (n=10, combined from 2 experiments). L) Numbers of GI organoids grown from colonic crypt isolates and enumerated at D5 from mice as per J) and K) (n=6, combined from 2 experiments). Data are presented as mean±SEM. p values calculated using two tailed Mann-Whitney T test. Survival calculated using Log-rank Mantel-Cox test. *p<0.05, **p<0.01.

FIG. 5. IFNλ treatment produces a proliferative phenotype in GI stem cells. Recombinant PEGylated lambda interferon (PEG-rIL-29, 5 μg) or PBS was given I.P. for three days prior to gut harvest and evaluation of GI epithelial function. A) Numbers, B) Representative images and C) Size of GI organoids grown from colonic crypt isolates (n=7 per group, combined from 3 experiments). D) Numbers of small intestinal organoids. (n=5, combined from 3 experiments). E) Number of stem cells (CD45.2⁻/EpCAM⁺/GFP⁺) isolated from digested gut of Lgr5-EGFP-IRES^{creERT2} mice (n=5, combined from 3 experiments). F) Number of organoids grown from FACS sorted single stem cells (n=4, combined from 3 experiments). G) Number of colonic organoids from WT and IL-22^{-/-} mice treated with PBS or PEG-rIL-29 (n=4, combined from 2 experiments). H) Number of organoids from WT and Ifnar^{-/-} mice treated with PBS or PEG-rIL-29 (n=6, combined from 3 experiments). I) RNAseq from sort purified single colonic epithelial cells and stem cells derived from either PEG-rIL-29 or PBS treated mice. Heat map showing top 300 genes significantly differentially expressed in colonic epithelial cells and stem cells after in vivo PEG-rIL-29 vs PBS treatment (2420 genes total). Expression of the same genes from intestinal stem cells derived from PBS or IL-29 treated mice included for comparison (n=5 per group). J Normalized mRNA transcript counts for Ifnlr1, Il10rb, Ifnar1 and Ifnar2 in colonic epithelial cells (Lgr5⁻) and stem cells (Lgr5⁺). K) Functional enrichment analysis of differentially expressed genes: Canonical Pathway enrichment analysis (log 2 Fold-change>|0.58| and adj. p-value< 0.05) across PEG-rIL-29-treated Lgr5⁺ and Lgr5⁻ samples relative to genotype-matched PBS-treated samples using IPA. Enrichment of canonical pathways associated with immune responses (left) and regulation of cellular proliferation (right). Bubbles represent significant pathway enrichment, as determined by Fisher exact test. Bubble diameter represents the −log 10 P value as determined by Fisher's exact test. Crosses signify a lack of significant pathway enrichment. Color indicates predicted pathway activation (red) or predicted inhibition (blue). White bubbles represent significant functional enrichment of pathways with no available prediction patterns. L) Venn diagram of overlap of DE genes in Lgr5+ and Lgr5− cells as for K). Data are presented as mean±SEM. p values calculated using two tailed Mann-Whitney T test. *p<0.05, **p<0.01, ***p<0.001. For RNAseq differentially regulated genes were determined after filtering for genes with >5 cpm and fold change differences of >1.2 and corrected p values (FDR) of <0.05.

FIG. 6 Predicted activation state of PEG-rIL-29 treated Lgr5⁺ and Lgr5⁻ sorted GI epithelial cells. A) Cytokines, B) Transcriptional regulators, and C) Kinases with significantly associated transcriptional changes after PEG-IL29-treated Lgr5⁺ and Lgr5⁻ cells using IPA. Bubble plot representation of significant enrichment scores (activation z-score>|2|) in at least one treatment condition. Color indicates predicted activation (red) or predicted inhibition (green) and bubble diameter represents the −log 10 P value as determined by Fisher's exact test. Crosses signify a lack of significant activation scores at specific timepoints.

FIG. 7. IFNλ treatment protects from GVHD within the GI tract. PEG-rIL-29 or PBS was given as previously described on day −2, −1 and day 0 to BMT recipients. A) Survival by Kaplan-Meier analysis of B6 recipients transplanted with BALB/c BM+T cells (n=10, combined from 2 experiments). B) Survival by Kaplan-Meier analysis of B6D2F1 recipients transplanted with B6 BM+T cells (n=10, combined from 2 experiments). C) Serum IFNγ and IL-6 at day 4 after BMT as in A (n=15, combined from 3 experiments). D) Representative images of colon and small intestine (SI) at D7 post-BMT E) Semi-quantitative GVHD histopathology scores at D7 post-BMT (n=10, combined from 2 experiments). F) Paneth cell numbers. G) Representative images of proliferation in colon and SI using Ki-67 at day 7 after BMT H) Quantification of Ki-67 expression at day 7 post-BMT in colon (n=10, combined from 2 experiments) and I) in SI as in H. J) B6 recipients were transplanted with BALB/c BM±T cell and crypt isolates obtained 7 days later. Colon organoids were quantified (n=4, combined from 2 replicate experiments). K) Numbers of GFP⁺ Lgr5⁺ cells isolated at day 7 post-BMT from PBS or PEG-rIL-29 treated recipients (n=6, combined from 2 experiments). L) Representative immunoflourescent images as for K). M) Exemplar dual immunoflourescent images as for K with secondary staining for GFP and Ki-67. N) B6D2F1 recipients were treated with PBS or PEG-rIL-29, then transplanted with BM±T cells from B6.WT donors together with recipient type BCR-ABL nup98hoxA9 leukemia expressing GFP. The percentage of GFP⁺ leukemia cells was determined in peripheral blood thereafter. (n=10, combined from 2 experiments). O) Relapse and death from leukaemia in recipients as transplanted in R). Data are presented as mean±SEM. p values calculated using two tailed Mann-Whitney T test. Survival calculated using Log-rank Mantel-Cox test. *p<0.05, **p<0.01, ***p<0.001, ****p<0.0001.

FIG. 8. Histological examination of naïive WT and Ifnlr1^{-/-} GI tissues. A) Representative H&E stained images of colon and SI from naïve WT and Ifnlr1^{-/-} mice. B) Pre-transplant weight of WT and Ifnlr1^{-/-} mice; WT and Ifnlr1^{-/-} were not aged matched (WT n=14, Ifnlr1^{-/-} n=20, combined from 2 experiments). C) Representative period acid-schiff stained images of colon and SI from naïve WT and Ifnlr1^{-/-} mice demonstrating goblet cells. D) Enumeration of goblet cells in WT and Ifnlr1^{-/-} mice (n=3). E) Representative images of colon and SI from naïve WT and Ifnlr1^{-/-} mice stained by immunohistochemistry for lysozyme demonstrating Paneth cell numbers. F) Enumeration of Paneth cells in WT and Ifnlr1^{-/-} mice (n=3).

FIG. 9. GVL capacity of WT and Ifnlr1−/− donor grafts against leukaemic cell lines. A) Ifnlr1^{-/-} BM+T BMT grafts retain GVL capacity. Balb/c recipients were transplanted with BM±T cells from WT or Ifnlr1^{-/-} donors, together with recipient type MLL-AF9 leukemia expressing GFP. The absolute number of GFP+ leukemia cells in peripheral blood was determined thereafter (n=18, combined from 3 replicate experiments). B) B6D2F1 recipients were transplanted with BM±T cells from WT or Ifnlr1^{-/-} donors together with recipient type BCR-ABL nup98hoxA9 leukemia expressing GFP. The absolute number of GFP+ leukemia cells in peripheral blood was determined thereafter (n=12, combined from 2 experiments). Data are presented as mean±SEM.

FIG. 10. Sort strategy for isolation of Lgr5+ cells from GI tissues. A) Representative images from FACS sorting strategy for isolation of GFP+ Lgr5+ cells from colonic tissue samples. B) Expression of intestinal stem cell markers from GFP+ and GFP− sorted fractions by RNA sequencing (n=5, combined from 4 experiments). Data are presented as mean±SEM. p values calculated using the Benjamini-Hochberg procedure corrected for multiple comparisons. ****p<0.0001.

FIG. 11. Canonical Pathway enrichment analysis for differentially expressed genes across PEG-rIL-29-treated Lgr5+ and Lgr5− samples relative to genotype-matched PBS-treated samples. A) Representation of all canonical pathways found to be enriched in the dataset. Bubbles represent significant pathway enrichment, as determined by Fisher exact test. Bubble diameter represents the −log 10 P value as determined by Fisher's exact test. Crosses signify a lack of significant pathway enrichment. Colour indicates predicted pathway activation (red) or predicted inhibition (blue). White bubbles represent significant functional enrichment of pathways with no available prediction patterns. Data are presented as log 2 Fold-change>|0.58| and adj. p-value<0.05)

### DETAILED DESCRIPTION

The present invention is at least partly predicated on the surprising discovery that signalling by way of the IFNλ receptor 1 (IFNLR1) can influence the progression of GVHD, particularly in the gastrointestinal tract. In this regard, the present invention is further at least partly predicated on the surprising discovery that agonists or ligands of the IFNLR1, such as IL-29, exert a protective and/or proliferative effect on intestinal stem cells, which can be utilised to preserve the gastrointestinal epithelial lining.

In a first aspect, the invention provides a method of preventing or treating a gastrointestinal disease, disorder or condition in a subject, said method including the step of administering to the subject a therapeutically effective amount of an IFNλ receptor agonist to thereby prevent or treat the gastrointestinal disease, disorder or condition in the subject.

It is envisaged that the present method of treating the gastrointestinal disease, disorder or condition may be prophylactic, preventative or therapeutic and suitable for treatment of gastrointestinal diseases, disorders or conditions in mammals, particularly humans. As used herein, “treating” ,“treat” or “treatment” refers to a therapeutic intervention, course of action or protocol that at least ameliorates a symptom of the gastrointestinal disease, disorder or condition after the disease, disorder or condition and/or its symptoms have at least started to develop. As used herein, “preventing”, “prevent” or “prevention” refers to therapeutic intervention, course of action or protocol initiated prior to the onset of the gastrointestinal disease, disorder or condition and/or a symptom thereof so as to prevent, inhibit or delay or development or progression of the gastrointestinal disease, disorder or condition or the symptom.

The term “therapeutically effective amount” describes a quantity of a specified agent, such as of an IFNλ receptor agonist, sufficient to achieve a desired effect in a subject being treated with that agent. For example, this can be the amount of a composition comprising one or more IFNλ receptor agonists described herein, necessary to reduce, alleviate and/or prevent a gastrointestinal disease, disorder or condition, inclusive of gastrointestinal GVHD and recurrence thereof. In some embodiments, a “therapeutically effective amount” is sufficient to reduce or eliminate a symptom of a gastrointestinal disease, disorder or condition. In other embodiments, a “therapeutically effective amount” is an amount sufficient to achieve a desired biological effect, for example an amount that is effective to decrease the severity of or prevent a gastrointestinal disease, disorder or condition.

Ideally, a therapeutically effective amount of an agent is an amount sufficient to induce the desired result without causing a substantial cytotoxic effect in the subject. The effective amount of an agent useful for reducing, alleviating and/or preventing a gastrointestinal disease, disorder or condition will be dependent on the subject being treated, the type and severity of any associated disease, disorder and/or condition (e.g., the location of any associated disease), and the manner of administration of the therapeutic composition.

In one embodiment, the gastrointestinal disease, disorder or condition is at least partly characterized by gastrointestinal epithelial or epithelial cell damage and/or loss. To this end, it will be understood that gastrointestinal diseases, disorders and conditions can involve damage and/or at least partial loss of the gastrointestinal epithelial layer during initiation and/or progression thereof. Associated with such damage, subjects may be at risk of fibrosis and ulceration. The assessment and/or detection of gastrointestinal epithelial damage may be achieved by any method known in the art, such as endoscopy, colonoscopy, biopsy, histological analysis and faecal analysis.

The term “gastrointestinal epithelium” as used herein refers to epithelial tissues of the gastrointestinal tract, such as the oesophagus, the stomach, the small intestine (e.g., duodenum, jejunum, ileum) and/or the large intestine (e.g., caecum, colon). It may further include other structures involved in the gastrointestinal function of the body including the lower portion of the body, rectum and anus. To this end, the gastrointestinal disease, disorder or condition may affect the entire, or substantially the entire, gastrointestinal tract of a subject or alternatively may only affect one or more specific regions or portions thereof.

A “gastrointestinal epithelial cell” or “gut epithelial cell” as referred to herein refers to any epithelial cell on the surface of the gastrointestinal mucosa that faces the lumen of the gastrointestinal tract, including, for example, an epithelial cell of the stomach, an intestinal epithelial cell, a colonic epithelial cell, and the like.

The gastrointestinal or intestinal epithelium is a highly specialized tissue that lines the gastrointestinal tract and is critical to gut function, enabling both digestion and nutrient absorption, and provides both physical and chemical barriers to the gut microbiota. This gut barrier function also serves an important immunological role in preventing unwanted responses to microbes, but enables surveillance and appropriate immune control of infection. Intestinal epithelial cells (IECs) have a very high turnover rate (with replacement occurring every 4-5 days) and this continuous renewal is driven by long-lived intestinal stem cells (ISCs). This regenerative process is critical to maintenance of the intestinal epithelium, damage repair and preservation of gut-barrier integrity. Intestinal epithelium damage can occur in a range of settings including infection, chronic inflammation, autoimmunity as well as chemo/radiotherapy, and subsequent loss of barrier function can lead to further damage through feed-forward inflammatory responses. Damage to ISCs is particularly problematic due to their important regenerative role in forming intestinal epithelial cells.

Suitably, the gastrointestinal disease, disorder or condition is selected from the group consisting of an inflammatory intestinal disease, an infection, an autoimmune disease, such as autoimmune enterocolitis, radiation-induced intestinal damage, immunotherapy-induced intestinal damage (e.g., immunotherapy-related enterocolitis), an immune deficiency, a haematological malignancy, chemotherapy-induced intestinal damage (e.g., chemotherapy-related enterocolitis), graft versus host disease (GVHD), and any combination thereof.

As used interchangeably herein, the terms “inflammatory intestinal disease”, “inflammatory bowel diseases” or “IBD” includes art-recognized forms of a group of related inflammatory gastrointestinal conditions. Several major forms of IBD are known, such as Crohn's disease (regional bowel disease, inclusive of inactive and active forms) and ulcerative enterocolitis or colitis (inclusive of inactive and active forms). In addition, IBD encompasses irritable bowel syndrome, microscopic enterocolitis, lymphocytic-plasmocytic enteritis, coeliac disease, collagenous enterocolitis, lymphocytic enterocolitis and eosinophilic enterocolitis. Other less common forms of IBD include indeterminate enterocolitis, infectious enterocolitis (viral, bacterial or protozoan, e.g. amoebic enterocolitis) (e.g., Clostridium dificile enterocolitis), pseudomembranous enterocolitis (necrotizing enterocolitis), ischemic inflammatory bowel disease, Behcet's disease, sarcoidosis, scleroderma, IBD-associated dysplasia, dysplasia associated masses or lesions, and primary sclerosing cholangitis.

Accordingly, it will be appreciated that IBD represents chronic, inflammatory diseases of the gastrointestinal tract. IBD can be characterized by abdominal pain, diarrhoea (often bloody), a variable group of “extra-intestinal” manifestations (such as arthritis, uveitis, skin changes, etc.) and the accumulation of inflammatory cells within the small intestine and/or colon. Additional signs or symptoms of IBD include malabsorption of food, altered bowel motility, infection, fever, rectal bleeding, weight loss, signs of malnutrition, perianal disease, abdominal mass, and growth failure, as well as intestinal complications such as stricture, fistulas, toxic megacolon, perforation, and cancer, and including endoscopic findings, such as friability, aphthous and linear ulcers, cobblestone appearance, pseudopolyps and rectal involvement.

In a specific embodiment, the gastrointestinal disease, disorder or condition is or comprises GVHD. It will be appreciated that the term “graft-versus-host disease” or “GVHD” refers to a complication of allogeneic stem cell transplantation. In GVHD, donor hematopoietic stem cells recognize the transplant recipient as foreign and attack the patient's tissues and organs, which can impair the tissue or organ's function and/or cause it to fail. As used herein, GVHD includes, for example, acute GVHD and/or chronic GVHD. Acute and chronic GVHD are a major cause of morbidity and mortality among hematopoietic stem cell transplant recipients. Symptoms can include skin rash, intestinal problems similar to colitis or enterocolitis, and liver dysfunction. Accordingly, further non-limiting examples of GVHD include gastrointestinal GVHD, cutaneous GVHD and hepatic GVHD.

As used herein, the terms “gastrointestinal graft vs. host disease” and “GI-GVHD” refer to damage caused by donor immune cells to host tissue of the stomach and intestine, including the small intestine, large intestine and colon, which can cause loss of appetite, nausea, vomiting, or diarrhoea as part of GVHD, either acute or chronic. In severe cases, GI-GVHD can cause pain in the abdomen and bleeding in the stomach or intestines. To this end, GVHD in the subject is suitably characterised by or comprises colonic epithelial or epithelial cell damage and/or loss.

Accordingly, in certain embodiments, the method of the present aspect includes the further step of determining whether the subject has a gastrointestinal disease, disorder or condition, such as GVHD, or is at risk of developing a gastrointestinal disease, disorder or condition prior to administration of the IFNλ receptor agonist. The terms “determining”, “measuring”, “evaluating”, “assessing” and “assaying” are used interchangeably herein and may include any form of measurement known in the art.

The term “gastrointestinal infection” or “intestinal infection” as used herein refers to a viral, bacterial, fungal or parasitic infection in the gastrointestinal tract or intestine of said animal. Said infection may cause clinical or subclinical enteric disease in said animal.

The terms “autoimmune disease” or “autoimmune disorder” are used herein interchangeably and refer to any disease or disorder that occurs when the body's immune system erroneously attacks and destroys healthy body tissue, such as the gastrointestinal tract. According to one embodiment, the autoimmune disease is or comprises a gastrointestinal autoimmune disease or digestive autoimmune disease, such as ulcerative colitis, Crohn's disease, celiac disease, irritable bowel syndrome or autoimmune hepatitis. The terms “gastrointestinal autoimmune disease” and “digestive autoimmune disease” are used herein interchangeably and refer to autoimmune disease of a gastrointestinal tract of a subject.

It will be appreciated that radiation-induced damage to epithelial cells can lead to inflammation, epithelial cell apoptosis and death, and/or loss of epithelial cell (e.g., intestinal) barrier function. The mechanism by which radiation, e.g., abdominal radiation, causes such effects on cells (e.g., epithelial cells) is complex. Radiation has been shown to affect various components of the local intestinal response to injury and microbial invasion that includes rapid depletion of mucus, immune impairment, oxidant-mediated epithelial cell injury, and radiation-induced epithelial cell apoptosis. Exemplary symptoms include nausea, vomiting, diarrhoea, dehydration, electrolytic imbalance, loss of digestion ability, bleeding ulcers and sepsis.

It will be appreciated that the present aspect may include administration of one or more further treatments or therapeutic agents in addition to the IFNλ receptor agonist, as are known in the art.

In one particular embodiment, the subject has or is to be administered an immunosuppressive agent. In alternative embodiments, the subject has not or is not to be administered an immunosuppressive agent. In this regard, treatment of the subject with the IFNλ receptor agonist may preclude the need, at least partly, for further treatment with an immunosuppressive agent.

The term “immunosuppressive agent” as used herein for adjunct therapy refers to substances that act to suppress or mask the immune system of the host into which a graft, such as haematopoietic stem cells, is being transplanted. This would include, for example, agents that suppress cytokine production, downregulate or suppress self-antigen expression, or mask the MHC antigens. Examples of such agents include non-steroidal anti-inflammatory drugs (NSAIDs); ganciclovir, tacrolimus, steroids such as corticosteroids, glucocorticoids or glucocorticoid analogues, e.g., prednisone, methylprednisolone, and dexamethasone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens); anti-idiotypic antibodies for MHC antigens and MHC fragments; cyclosporin A; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor (TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; streptokinase; transforming growth factor-beta (TGF-beta); streptodomase; chlorambucil; deoxyspergualin; rapamycin; and biologic agents that interfere with T cell helper signals.

In other embodiments, the IFNλ receptor agonist is administered to the subject prior to, simultaneously with and/or subsequent to administration of a transplant. Suitably, the transplant is a haematopoietic stem cell transplant.

The terms “haematopoietic stem cell transplant” and “haematopoietic cell transplant” are used interchangeably herein and refer to the transplantation of cells or stem cells derived at least partly from bone marrow, peripheral blood, placental blood and/or umbilical cord blood. Hematopoietic stem cells are stem cells that generate the red blood cells, white blood cells, and platelets that make up blood. Such a transplant may be autologous, involving cells collected from the subject in question, or allogeneic, utilizing cells from a donor other than the subject. It will be appreciated that a haematopoietic stem cell transplant may include the use of high dose chemotherapy or radiotherapy, including whole-body irradiation, to eliminate diseased cells, such as leukemic immune cells, prior to introduction of the transplanted haematopoietic stem cells. By way of example, patients with leukemia do not produce normal functioning blood cells and transplanting healthy haematopoietic stem cells from a donor with a histocompatibility antigen type (HLA) that is preferably similar to that of the patient with leukemia (and with or without chemotherapy or radiotherapy) into the leukemic patient can then produce blood cells that at least partly function normally.

In particular embodiments, the haematopoietic stem cell transplant can be an allogeneic bone marrow transplant, an umbilical cord blood transplant, a peripheral blood transplant, a placenta-derived blood transplant and/or a haematopoietic stem cell line such as conditionally immortalized haematopoietic stem cells. It will be appreciated that the transplant may be purified or at least partially purified prior to administration to the subject.

The skilled artisan will appreciate that haematopoietic stem cell transplants can benefit subjects with a cancerous disease or a noncancerous disease. Nonlimiting examples of such diseases include acute lymphoblastic leukemia, adrenoleukodystrophy, aplastic anaemia, bone marrow failure syndromes, chronic lymphoblastic leukemia, haemoglobinopathies, Hodgkin's lymphoma, immunodeficiencies, inborn errors of metabolism, multiple myeloma, myelodysplastic syndromes, neuroblastoma, non-Hodgkin's lymphoma, plasma cell disorders, POEMS syndrome and primary amyloidosis.

Suitably, the IFNλ receptor agonist does not modulate and/or preserves graft versus leukemia and/or graft versus tumour effects of the transplant. As used herein, the term “graft-versus-leukemia” or “GVL” or “graft-versus-tumour” or “GVT” refers to a beneficial therapeutic immune reaction of the transplanted immune cells, such as donor T lymphocytes, against the diseased bone marrow and/or residual cancer cells of the recipient subject.

In view of the above, in some embodiments, the IFNλ receptor agonist can be administered to the subject prior to, simultaneously with and/or subsequent to administration of a cytotoxic agent. In this regard, it is well established that gastrointestinal epithelial damage is a common side effect of many cytotoxic agents. Moreover, in the case of an allogeneic haematopoietic stem cell transplant, one or more cytotoxic agents, such as a chemotherapeutic agent and/or radiation can be administered to the subject to not only assist in removing any diseased or cancerous cells but also suppress the subject's immune system to allow the transplanted bone marrow to undergo a process called engraftment.

The term “cytotoxic agent” as used herein refers to a substance that inhibits or prevents a cellular function and/or causes cell death or destruction. Cytotoxic agents include, but are not limited to, radiotherapy, such as by way of radioactive isotopes (e.g., At²¹¹, I¹³¹, I¹²⁵, Y⁹⁰, Re¹⁸⁶, Re¹⁸⁸, Sm¹⁵³, Bi²¹², P³², Pb₂₁₂ and radioactive isotopes of Lu); chemotherapeutic agents; growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof and other anticancer agents as are known in the art.

In one particular embodiment, the cytotoxic agent is or comprises a chemotherapeutic agent. As generally used herein, the term “chemotherapy” or “chemotherapeutic agent” broadly refers to a treatment or agent with a cytostatic or cytotoxic agent (i.e., a compound) to reduce or eliminate the growth or proliferation of undesirable cells, such as cancer cells. Accordingly, the terms can refer to a cytotoxic or cytostatic agent used to treat a proliferative disorder, for example cancer. The cytotoxic effect of the agent can be, but is not required to be, the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect.

Exemplary chemotherapeutic agents include, but are not limited to, alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin, oxaliplatin, nedaplatin, triplatin tetranitrate, phenanthriplatin, picoplatin, satraplatin and lipoplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil (5-FU), fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, docetaxel and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukemias and blastomas, although without limitation thereto. In particular embodiments, the subject has a cancer, such as a liquid cancer or a blood cell cancer inclusive of lymphoid cancers and myelomonocytic cancers, such as those hereinbefore described, that is potentially or at least partly amenable to treatment by administration of a transplant, such as a haematopoietic stem cell transplant.

As used herein, the term “IFNλ receptor agonist” refers to any substance, compound or molecule capable of activating an IFNλ receptor. It may activate the IFNλ receptor directly or indirectly. For clarity, the term “IFNλ receptor agonist” may also include an IFNλ receptor activator that activates the IFNλ receptor directly or indirectly. The IFNλ receptor comprises subunits IL10R2 (CRF2-4, UniProt Accession No: Q08334) and IFNλR1 (IL28RA, CRF2-12; Uniprot Accession No: Q8IU57). Activation of the IFNλ receptor triggers Janus kinase activation (Jak1 and Tyk2) and phosphorylation and activation of the transcription factors STAT1, STAT2 and STAT3, and interferon-regulated transcription factors IRFs. Upon phosphorylation, STATs translocate into the nucleus to induce hundreds of genes altogether termed IFN-stimulated genes or ISGs. Accordingly, an IFNλ receptor agonist is suitably capable of stimulating one or more of these downstream signalling events via the IFNλ receptor.

It is envisaged that the IFNλ receptor agonist or activator can be any as are known in the art. For example, the agonist/activator of IFNλ receptor may be a small molecule, an antibody or an antibody fragment, a synthekine, a peptide, a polynucleotide expressing IFNλ, an IFNλ ligand or molecule or a fragment, variant or derivative thereof. An activator of an IFNλ receptor may also be an agent that triggers an increase of endogenous IFNλ in a subject after administration of the agent to the subject.

Lambda IFNs (IFNλs), type III IFNs or IL-28/29 constitute one of the most recent additions to the interferon family (Lazear et al., 2015). They consist of four members in humans (IFNλ1/IL-29, Uniprot Accession No. Q8IU54; IFNλ2/IL-28A, Uniprot Accession No. Q8IZJ0; IFNλ3/IL-28B, Uniprot Accession No. Q8IZI9; IFNλ4, Uniprot Accession No. K9M1U5) and although a broad range of pathogens are capable of inducing IFNλs, the cellular sources of IFNλs are relatively limited and include epithelial cells, especially at mucosal interfaces, conventional and plasmacytoid dendritic cells, and monocytes.

The IFNλ receptor agonist may further encompass fragments, variants, derivatives and functional equivalents of IFNλ1/IL-29, IFNλ2/IL-28A, IFNλ3/IL-28B and IFNλ4.

By the term “variants”, substantially similar amino acid sequences of wild-type IFNλ ligands or molecules are intended. The IFNλ polypeptides in accordance with the present invention may be altered in various ways including amino acid substitutions, deletions, truncations and insertions. They may also undergo posttranslational modification. Novel proteins having properties of interest may be created by combining elements and fragments of IFNλ proteins or their receptors, as well as with other proteins. This may lead to greater stability or greater bioavailability in the GI tract, for example. Methods for such manipulations are generally known in the art. The IFNλ proteins encompass naturally occurring proteins as well as variant, truncated and modified forms or derivatives thereof.

A “fragment” is a segment, domain, portion or region of a protein, such as an IFNλ ligand or cytokine described herein, which constitutes less than 100% of the amino acid sequence of the protein. In general, fragments may comprise up to 5, 6, 7, 8, 9, 10, 11, 12, 13, 14, 15, 16, 17, 18, 19, 20, 30, 40, 50, 60, 70, 80, 90, 100, 110, 120, 130, 140, 150, 200, 250, 300, 350, 400, 450, 550 or up to about 600 amino acids of an amino acid sequence. Suitably, the fragment contains or comprises a portion of an IFNλ ligand capable of binding and/or activating an IFNλ receptor.

As used herein, “derivative” proteins have been altered, for example by conjugation or complexing with other chemical moieties, by post-translational modification (e.g. phosphorylation, acetylation and the like), modification of glycosylation (e.g. adding, removing or altering glycosylation) and/or inclusion of additional amino acid sequences as would be understood in the art. In particular embodiments, the IFNλ receptor agonist comprises an IFNλ derivative that has been modified to extend the serum half-life thereof, such as by the addition of one or more PEG groups or albumin binding domains (ABD). In some embodiments, this may lead to greater stability or greater delivery and bioavailability in the GI tract, for example.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g. polyhistidine) fusion partners, maltose binding protein (MBP), Protein A, glutathione S-transferase (GST), fluorescent protein sequences (e.g. GFP), epitope tags such as myc, FLAG and haemagglutinin tags. For example, the IFNλ receptor agonist may comprise an IFNλ ligand that is pegylated (e.g., pegylated recombinant IL29), in particular monopegylated, and/or conjugated with a polyalkyl oxide moiety. Such fragments, variants and derivatives will suitably continue to possess the desired IFNλ activity (i.e., functional variants, fragments or derivatives). By way of example, exemplary IL-29 variants and fragments are provided in US20110243888, which is incorporated by reference herein.

Other derivatives contemplated by the invention include, but are not limited to, modification to side chains, incorporation of unnatural amino acids and/or their derivatives during peptide, or protein synthesis and the use of crosslinkers and other methods which impose conformational constraints on the immunogenic proteins, fragments and variants of the invention.

In some embodiments, IFNλ receptor agonist variants or derivatives may include the IFNλ receptor agonist being in a more stabilised form or in a prodrug form, preferably suited for oral delivery or otherwise for delivery to the GI tract, for example. In some embodiments, IFNλ receptor agonist variants or derivatives may be capable of generating an orally bioavailable cytokine targeted to the GI tract. In some embodiments, the IFNλ receptor agonist variants or derivatives may be suitable for oral administration. In some embodiments, IFNλ receptor agonist variants or derivatives may include the IFNλ receptor agonist being in a prodrug form, for providing greater bioavailability of the IFNλ receptor agonist in the GI tract, for example.

Suitably, the IFNλ receptor agonist is administered to the subject as a pharmaceutical composition comprising a pharmaceutically-acceptable carrier, diluent or excipient.

By “administering” or “administration” is meant the introduction of an IFNλ receptor agonist described herein into an animal subject by a particular chosen route.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In a further aspect, the invention provides a method of promoting survival and/or proliferation of an intestinal cell, said method including the step of contacting the intestinal cell with an IFNλ receptor agonist under conditions to promote survival and/or proliferation of the intestinal cell.

Exemplary intestinal cells include intestinal stem cells, enterocytes, goblet cells, enteroendocrine cells, Paneth cells, microfold or M cells, cup cells, Tuft cells Suitably, the intestinal cell is or comprises an intestinal stem cell (ISC). As used herein, the term “intestinal stem cell” refers to a multipotent stem cell, such as those intestinal cells that express or are positive for one or more of Lgr5, ALCAM, ASCL2, BMI1, DCLK1, EPHB2, HOPX, Igfbp4, Itgb1, Lrig1, mTert, Musashi-1, OLFM4, PHLDA1, Prom1, PW1, Smoc2, Sox9 and TNFRSF19.

It is envisaged that the present method can be performed in vitro. In this regard, the present method may be utilised to generate intestinal cells or organoids, such as intestinal stem cells or organoids thereof, that may be subsequently administered to a subject as a prophylactic or therapeutic agent for a gastrointestinal disease, disorder or condition. In other embodiments, the intestinal cells, tissues and/or organoids described herein may be useful for toxicity screening or for in vitro drug safety testing. By way of example, some drugs used for therapeutic interventions can cause unexpected toxicity in intestinal cells or tissue, often leading to significant morbidity and disability.

In alternative embodiments, the method of the present aspect can be performed in vivo. To this end, the present method relates to the promotion of survival and/or proliferation of an intestinal cell in a subject, which includes the step of administering to the subject a therapeutically effective amount of an IFNλ receptor agonist to thereby promote survival and/or proliferation of the intestinal cell therein. To this end, the IFNλ receptor agonist may be administered to a subject to promote preservation of the gastrointestinal epithelial layer and/or promote proliferation and/or regeneration thereof.

In one embodiment, the IFNλ receptor agonist is administered prior to, simultaneously with and/or subsequent to administration of a cytotoxic agent, such as those hereinbefore described, to the subject. To this end, the cytotoxic agent suitably is or comprises a chemotherapeutic agent and/or a radiotherapy.

Suitably, the subject has or is suffering from a gastrointestinal disease, disorder or condition, such as inflammatory intestinal disease, autoimmune disease, immunotherapy-induced intestinal damage, an immune deficiency, a haematological malignancy, chemotherapy-induced intestinal damage, radiation-induced intestinal damage, GVHD, and any combination thereof.

Suitably, the IFNλ receptor agonist is selected from the group consisting of IFN-λ1 (IL-29), IFN-λ2 (IL-28A), IFN-λ3 (IL-28B), IFN-λ4, including variants and derivatives thereof and any combination thereof. More particularly, the IFNλ receptor agonist suitably is or comprises recombinant IL-29, such as pegylated recombinant IL-29.

In another aspect, the invention provides a method of performing a transplant in a subject in need thereof, said method including the step of administering to the subject a therapeutically effective amount of an IFNλ receptor agonist prior to, simultaneously with and/or subsequent to administration of the transplant to the subject.

In certain embodiments, the transplant is or comprises a hematopoietic stem cell transplant, such as those described herein. In one particular embodiment, the transplant is or comprises a bone marrow transplant.

In one embodiment, the present method includes the further step of administering the transplant to the subject.

Suitably, the IFNλ receptor agonist is that described herein, such as IFN-λ1 (IL-29), IFN-λ2 (IL-28A), IFN-λ3 (IL-28B), IFN-λ4, including fragments, variants and derivatives thereof and any combination thereof. More particularly, the IFNλ receptor agonist suitably is or comprises recombinant IL-29, such as pegylated recombinant IL-29.

Suitably, the IFNλ receptor agonist and/or the transplant are administered to a subject as a pharmaceutical composition comprising a pharmaceutically-acceptable carrier, diluent or excipient. In this regard, any dosage form and route of administration, such as those provided herein, may be employed for providing a subject with the composition of the invention.

In a further aspect, the invention relates to an IFNλ receptor agonist for use in:

(a) preventing or treating a gastrointestinal disease, disorder or condition;

(b) promoting survival and/or proliferation of an intestinal cell; and/or

(c) performing a transplant;

in a subject.

In a related aspect, the invention resides in use of an IFNλ receptor agonist in the manufacture of a medicament for:

(a) preventing or treating a gastrointestinal disease, disorder or condition;

(b) promoting survival and/or proliferation of an intestinal cell; and/or

(c) performing a transplant;

in a subject.

For the two aforementioned aspects, the IFNλ receptor agonist is suitably selected from the group consisting of IFN-λ1 (IL-29), IFN-λ2 (IL-28A), IFN-λ3 (IL-28B), IFN-λ4, including fragments, variants and derivatives thereof and any combination thereof. More particularly, the IFNλ receptor agonist suitably is or comprises recombinant IL-29, such as pegylated recombinant IL-29.

Suitably, the gastrointestinal disease, disorder or condition of the two aforementioned aspects is that hereinbefore described. In one embodiment, the gastrointestinal disease, disorder or condition is selected from the group consisting of an inflammatory intestinal disease, an infection, an autoimmune disease, immunotherapy-induced intestinal damage, an immune deficiency, a haematological malignancy, chemotherapy-induced intestinal damage, radiation-induced intestinal damage, GVHD, and any combination thereof.

Suitably, the intestinal cell of the two aforementioned aspects is or comprises an intestinal stem cell, such as that described herein.

Suitably, the transplant of the two aforementioned aspects is that hereinbefore described and more particularly is or comprises a haematopoietic stem cell transplant.

With respect to the aforementioned aspects, the term “subject” includes but is not limited to mammals inclusive of humans, performance animals (such as horses, camels, greyhounds), livestock (such as cows, sheep, horses) and companion animals (such as cats and dogs). In one particular embodiment, the subject is a human.

So that preferred embodiments of the invention may be fully understood and put into practical effect, reference is made to the following non-limiting examples.

### EXAMPLE 1

Despite recent recognition of the important and unique immunology of the gastrointestinal (GI) mucosal barrier, the factors controlling the interface between the luminal microbiome and local immune cell populations in driving lethal acute GVHD (aGVHD) remain poorly defined (Jenq et al., 2012; Simms-Waldrip et al., 2017; Zeiser et al., 2016). The manipulation of targets preferentially active at this interface constitutes a rational therapeutic strategy that avoids the detriment of broad immune suppression. Type II interferons (i.e. IFNγ) are known to be directly pathogenic to the GI tract (Burman et al., 2007). In contrast, previous work by Robb (Robb et al., 2011) and Fischer (Fischer et al., 2017) identified a protective role for type I IFNs in the GI tract, although their relative importance on the initiation of MHC class II-dependent immunity by hematopoietic antigen presenting cell (APC) versus direct effects on the GI tract remain unclear. Recently, type III IFNs (lambda interferon (IFNλ)) (Kotenko et al., 2002; Sheppard et al., 2002) are increasingly recognized as the dominant IFN subtype driving innate immune responses at mucosal barriers (Baldridge et al., 2015; Galani et al., 2017; Lin et al., 2016) that can be disrupted by GVHD. We thus sought to define the role for type III, or IFNλ in the setting of transplantation. The IFNλ receptor is a heterodimer containing a specific IFNλ receptor chain (Ifnlr1) and the IL-10RB subunit (Kotenko et al., 2002). Genes for lambda IFNs and Ifnlr1 appear to be evolutionarily conserved, with humans possessing genes for 4 ligands IFNL1 (IL-29), IFNL2 (IL-28A), IFNL3 (IL-28B) and IFNL4. Mice express IFNL2 and 3, whilst IFNL1 and IFNL4 exist as pseudogenes (Hamming et al., 2013; Lasfar, 2006; Prokunina-Olsson et al., 2013). IFNλ shares downstream signalling pathways with type I IFNs and receptor ligation triggers phosphorylation of STAT 1, 2, 3 and 5 (Dumoutier et al., 2003; Kotenko et al., 2002). However, in contrast to the ubiquitous expression of type I IFN receptors, Ifnlr1 is preferentially expressed in mucosal epithelia tissues, especially in the GI and respiratory tracts (Galani et al., 2017; Lin et al., 2016; Sommereyns et al., 2008). Compartmentalization of IFNλ cytokine effects within the GI tract, by virtue of the restricted expression of the cognate receptor, offers the potential to impact GI tract homeostasis with limited effects on systemic GVL and immunity.

The importance of lambda IFNs in mediating innate viral defence was initially demonstrated in hepatitis C infection (Ge et al., 2009), where increased rates of spontaneous viral remission and improved responses to therapy were shown in patients with polymorphisms in IFNλ gene loci (Tanaka et al., 2009). IFNλ-dependent defence from gastro-tropic viruses such as norovirus and rotavirus is apparent (Baldridge et al., 2015; Lin et al., 2016; Rocha-Pereira et al., 2018). Homeostatic and protective paracrine functions were recently described (Galani et al., 2017) and demonstrate an anti-inflammatory viral response in respiratory epithelia. While anti-inflammatory effects in other cells including neutrophils have also been demonstrated (Blazek et al., 2015; Broggi et al., 2017), the function of IFNλ in immunopathology at mucosal surfaces unrelated to viral infections is largely unexplored. In the present Example, we document that IFNλ acts as an intestinal stem cell cytoprotectant and pre-BMT recipient treatment with IFNλ preserves intestinal stem cell function and mucosal integrity thereby attenuating GVHD. Together these data support the use of IFNλ as a strategy to prevent aGVHD within the GI tract. Since peg -rIL-29 is currently in late phase III clinical trials as an adjunctive therapy to treatment for hepatitis C (Muir et al., 2014; Muir et al., 2010), a rational strategy for rapid translation to the clinic can be envisioned.

### Materials and Methods

### Animals

Female adult C57BL/6 (B6.WT, H-2^{b}, CD45.2⁺), B6D2F1 (H-2^{b/d}, CD45.2⁺) and BALB/c (H-2^{d}, CD45.2⁺) mice (Mus musculus) of greater than 6 weeks of age were purchased from Animal Resources Centre, Canning Vale, Western Australia, Australia. Female adult B6.Ifnlr1^{-/-}, B6.Ifnar1^{-/-}, BALB/c^{Luc}, BALB/c CD45.1, B6.TEa^{Luc}, B6.Lgr5-EGFP-IRES^{creERT2}, Ifnlr1^{-/-}, NKp46^{Cre}, Ifnlr1^{fl/fl}, Lgr5-EGFP-IRES^{creERT2}.Ifnlr1^{fl/fl} and B6.IL-22^{-/-} of greater than 6 weeks of age were bred in-house at QIMR Berghofer Medical Research Institute in a pathogen-free animal facility. B6.Inflr1^{-/-}.Ifnar1^{-/-} were derived from B6.Ifnlr1^{-/-} and B6.Infar1^{-/-} crosses. All procedures were approved by the QIMR Berghofer Medical Research Institute animal ethics committee. Mice were housed in groups of up to 6 animals per cage for experiments other than co-housing, where cages of up to 20 mice were maintained. Separately housed and co-housed strains remained in these housing arrangements post-transplant.

Animals were housed in sterile micro-isolator cages and received acidified autoclaved water and were supplied with standard chow. Water was supplemented with enrofloxacin for the first two weeks post transplantation. No enrofloxacin was supplied in experiments where fecal microbial 16S sequencing was performed. Littermates of the same gender were randomly assigned to experimental groups. Lgr5-EGFP-IRES^{creERT2} mice were genotyped using the following primers in standard PCR conditions (15020 mutant reverse—CTGAACTTGTGGCCGTTTAC (SEQ ID NO:1), 26840 wild type reverse—GTCTGGTCAGAATGCCCTTG (SEQ ID NO:2), 8060 Common—CTGCTCTCTGCTCCCAGTCT (SEQ ID NO:3)). Tamoxifen (1 mg/day) was administered via the intraperitoneal route for 5 days, 2 weeks before transplant to induce tamoxifen-dependent Cre recombinase under the control of the Lgr5 promotor as indicated. Ifnlr1^{fl/fl} mice were genotyped using the following primers in standard PCR conditions forward: TCT GAC ATC CGC TCA GCA CCA A (SEQ ID NO:4), reverse: GGG CCC GCC CAA ATA TAA ACC (SEQ ID NO:5). Sample sizes based on estimates from initial and previously published results to ensure appropriate power.

### Allogeneic Stem Cell Transplantation

Donor mice were euthanized and bone marrow isolated from pelvis, femur and tibia as required and T cell depleted using complement where indicated. T cells were isolated from spleens and purified using magnetic separation. For all transplants 5×10⁶ T cells were introduced simultaneously with 10×10⁶ unmanipulated bone marrow cells for GVHD inducing grafts, and bone marrow only for T cell depleted grafts. Grafts were introduced via tail vein injection 24 hours after lethal irradiation given in two divided doses; 1000 cGy for B6 recipients, 1100 cGy for B6D2F1 recipients and 950 cGy for bone marrow chimeras. GVHD was assessed clinically using established scoring systems (Cooke et al., 1996) and mice with clinical scores>6 were sacrificed in accordance with institutional guidelines. For GVL experiments 1×10⁶ viable BCR-ABL nup98hoxA9 or MLL-AF9 transformed leukemia cells were introduced simultaneously with the graft.

For chimeric transplantation, WT or Ifnlr1^{-/-} recipients were transplanted with bone marrow only from Ifnlr1^{-/-} or WT donors in a syngeneic transplant establishing hematopoietic, non-hematopoietic cells, or both as unable to signal through Ifnlr1. WT to WT and Ifnlr1^{-/-} to Ifnlr1^{-/-} controls were included. Secondary MHC disparate transplantation with BALB/c bone marrow and T cells was then performed to evaluate post-transplant outcomes. Where necessary congenically marked BALB/c strains (CD45.1 and CD45.2) were used to allow identification of donor derived cells in mixed chimeras, or where the source of differing grafts was desired, including in in vivo cytotoxicity experiments. For NK cell depletion; anti-NK1.1 antibody derived from PK136 (ATCC® HB-191™) or IgG control was given at the dose of 1 mg on day −1 and at 0.5 mg on days +3 and +6 via the intraperitoneal route. PEG-rIL-29 treatment; 5 μg per day was delivered in 200 μL PBS via the intraperitoneal route either three days before harvest of GI tissues, or on days −2, −1 and 0 of transplantation.

### Histopathology

Samples were collected and fixed in 10% formalin, and transferred to 70% ethanol prior to paraffin embedding. 5 μm samples were cut and stained with Hematoxylin and Eosin to allow blinded semi-quantitative GVHD scoring using a published system (Hill et al., 1997) by an experienced anatomical pathologist. Paneth cell numbers were quantitated after immunohistochemical staining with horseradish peroxidase and anti-lysozyme antibody with enumeration performed electronically using Aperio ImageScope software (Version 12.3.2.8013) and the cytoplasmic staining algorithm. Ki67 was quantitated after immunohistochemical staining with horseradish peroxidase and enumeration performed using Aperio ImageScope (Version 12.3.2.8013) IHC nuclear algorithm. Three well sectioned areas devoid of processing artefact were chosen per slide and average % of positive cells per slide depicted.

### Immunofluorescence

Tissues were fixed with 4% paraformaldehyde, then placed in 30% sucrose prior to being frozen in Tissue-Tek OCT compound (Sakura Finetek). Sections (7 μm thickness) were treated with Background Sniper (Biocare Medical) and 2% BSA for 30 min and then stained with anti-GFP (Abcam ab290) and Ki-67 (DakoM7248) for 120 minutes at room temperature in the dark. After washing, sections were counterstained with DAPI for 5 min and cover-slipped with Vector Vectashield Hard Set mounting media. Images were taken with ×10 non-oil lens using a Zeiss 780-NLO Point Scanning Confocal Microscope with Zen software (Zen software).

### FITC Dextran

Seven days post-transplantation, mice were fasted of food and water for 4 hours prior to oral gavage with 8 mg of FITC labelled Dextran (MW 4 kDa, Sigma-Aldrich) in 200 μL of PBS. Peripheral blood was collected 4 hours later and serum separated. FITC-Dextran concentration in serum was determined using a Synergy H4 Fluorometer (Biotek) at excitation 485 nm and emission 535 nm.

### Cytokine Analysis

Serum IL-6, IL-17A, TNF and IFNγ were measured via murine Flex Array™ sets (BD Biosciences Pharmingen, San Diego, Calif., USA) according to manufacturer's instructions. Samples were acquired on a BD LSR Fortessa and analyzed using FCAP Array™ Software (BD Biosciences).

Serum IL-28A/B and SI and colon IL28-A/B were measured using R&D Systems Mouse IL-28A/B (IFN-lambda 2/3) DuoSet ELISA on samples obtained from either serum or mucosal intestinal homogenate as per Invitrogen. Briefly the mucosa was scraped from the underlying muscle layer with a glass slide. The cells of the mucosa were lysed with Tris EDTA (10 mM Tris-HCl, and 1 mM EDTA, pH 7.4) containing 0.05% sodium azide, 1% Tween-80, 2 mM PMSF, and 1 microgram per milliliter of each of the following protease inhibitors: aprotinin, leupeptin, and pepstatin A. The mucosa was homogenized (20 s). The homogenate was centrifuged (11,000×g, 10 minutes at 4° C.). The supernatant was collected. The supernatant was filtered (4.5 micron filter).

### qPCR

RT-qPCR was performed on RNA isolated from tissues isolated from naïve mice and mice post-transplant. Tissues were frozen in 500 μL Trizol and then mechanically homogenated. RNA was then extracted using QIAGEN RNeasy micro kit, was converted to cDNA and PCR performed using Taqman reagents. For Ifnlr1 Taqman Gene Expression Assay SM Mm00558035_m1 was used and for housekeeping SM Mm03024075_m1 (Hprt). For Reg3b, Reg3g, LysP and GAPDH primers were used with Sybr Green Supermix using standard PCR conditions on an ABI ViiA7 PCR machine. The primers used were; Reg3b F ACTCCCTGAAGAATATACCCT (SEQ ID NO:6) R GCTATTGAGCACAGARACGAG (SEQ ID NO:7), Reg3g F ATGCTTCCCCGTATAACCATCA (SEQ ID NO:8) R GGCCATATCTGCATCATACCAG (SEQ ID NO:9), Lysp F ATGGCTACCGTCGRGRCAAG (SEQ ID NO:10) R CGGTCTCCACGGTTGTAGTT (SEQ ID NO:11) and Gapdh F GACATGCCGCCTGGAGAAAC (SEQ ID NO:12) R AGCCCAGGATGCCCTTAGT (SEQ ID NO:13), all from Sigma Aldrich.

### Whole Animal and Organ Imaging

Expansion of luciferase expressing T cells was quantitated through measurement of luciferin-luciferase signal intensity using the Xenogen imaging system (Xenogen IVIS 100; Caliper Life Sciences, CA., USA). Fur on the ventral surfaces was shaved and mice were injected with 500 μg of luciferin subcutaneously and imaged 5 minutes later under continuous isoflurane-based anesthesia. After total body imaging mice were again injected with luciferin and then euthanized and single organs were isolated and imaged. For assessment of donor T cell expansion after transplant, BALB/c^{Luc} T cells were given at time of BMT. For assessment of APC function, expansion of TEa^{Luc} T cells in response to selected antigen presenting cells was performed at day 15 post-transplantation, with T cells given on day 12 post-transplant.

### Flow Cytometry and Antibody Staining

Single cell organ suspensions were prepared and all FACS enumeration was performed on a BD LSR Fortessa flow cytometer (BD Biosciences) unless otherwise specified. Post transplantation samples were incubated with 50 μL 24.G2 antibody for 5 minutes at room temperature to block Fc receptors. Samples and antibodies were incubated for 15 minutes at 4° C. in 50 μL of PBS containing 2% FBS. Proliferation assays were performed on T cells labelled with CFSE prior to transplantation. Cells were stained in 0.001 mM CFSE at 37° C. for 10 minutes, then washed and re-infused during BMT. Apoptosis assays were performed using BD Pharmingen Annexin V Apoptosis detection kit. Intracellular cytokine expression was measured after stimulation with PMA (5 μg/mL) and ionomycin (50 μg/mL) (Sigma-Aldrich) for 4 hours at 37° C. with Brefeldin A (BioLegend) included in the last 3 hours of culture. Cells were surfaced labelled, fixed and permeabilized (BD Biosciences—Cytofix/Cytoperm Kit) and stained with cytokine specific antibodies. Cell sorting was performed using a FACSAria II or III. Data were processed using FlowJo version 10 (FlowJo LLC). Detail of antibodies used is included in the Key Resources Table.

### Gut Digestion

For isolation and separation of cells from GI tract prior to FACS staining and/or sort separation the MACS Miltenyi Biotec Lamina Propria Dissociation Kit was used as per the manufacturer's instructions.

### Mixed Lymphocyte Reaction

BALB/c T cells were isolated from spleen and purified by magnetic bead selection. WT or Ifnlr1^{-/-} DC were isolated from spleen via density gradient and further purified by magnetic bead selection. Peritoneal macrophages were obtained by lavage. DC and macrophages were irradiated with 2100 cGy. Serial dilutions (20,000, 10,000, 5,000 and 0) of stimulator DC APC or 100,000 or 0 macrophage APC were plated with either CD4⁺ or CD8⁺ T cells at 200,000 cells per well in triplicate. After 4 days of culture, 100 μL per well of 1:1000 ³H-thymidine was added. 18 hours later proportionate inclusion of ³H-thymidine was measured scintigraphically.

### 16S Ribosomal Microbial Sequencing

DNA was extracted from 50-100 mg of fecal material using an initial bead beating step followed by extraction using the Maxwell 16 Research Instrument (Promega, USA) according to the manufacturer's protocol with the Maxwell 16 Tissue DNA Kit (Promega, USA). DNA concentration was measured using a Qubit assay (Life Technologies, USA) and was adjusted to a concentration of 5 ng/μl. The 16S rRNA gene encompassing the V6 to V8 regions was targeted using the 803F (5′-AAACTYAAAKGAATTGRCGG-3′ (SEQ ID NO:14)) and 1392R (5′-ACGGGCGGTGWGTRC-3′ (SEQ ID NO:15)) primers (Kunin et al., 2010) modified to contain Illumina specific adapter sequence (803F :5′ TCGTCGGCAGCGTCAGATGTGTATAAGAGACAGTTAGAKACCCBNGTAGTC 3′ (SEQ ID NO:16) and 1392wR:5′GTCTCGTGGGCTCGGGTCTCGTGGGCTCGGAGATGTGTATAAGAGACAG ACGGGCGGTGWGTRC3′ (SEQ ID NO:17)). Preparation of the 16S library was performed as described, using the workflow outlined by Illumina (#15044223 Rev. B). In the first stage, PCR products of ˜466 bp were amplified according to the specified workflow with an alteration in polymerase used to substitute Q5 Hot Start High-Fidelity 2X Master Mix (New England Biolabs, USA) in standard PCR conditions. Resulting PCR amplicons were purified using Agencourt AMPure XP beads (Beckman Coulter, USA). Purified DNA was indexed with unique 8 bp barcodes using the Illumina Nextera XT 384 sample Index Kit A-D (#FC-131-1002, Illumina, USA) in standard PCR conditions with Q5 Hot Start High-Fidelity 2X Master Mix. Indexed amplicons were pooled together in equimolar concentrations and sequenced on the MiSeq Sequencing System (Illumina, USA) using paired end sequencing with V3 300 bp chemistry at the Australian Centre for Ecogenomics according to manufacturer's protocol. Heat map includes OTUs identified as significantly different (p<0.001) between separately housed WT and Ifnlr1^{-/-} at week 4 where OTU relative abundance exceeds 2% in at least one sample. Each column includes scaled read counts for one mouse. Read counts normalized using metagenomeSeq.

### Organoids

Organoids were grown from crypt preparations harvested from small intestine or colon of naïve WT or Ifnlr1^{-/-} mice around 6-8 weeks of age. Crypt preparations were isolated following a recognized method (Miyoshi and Stappenbeck, 2013) with growth media derived from L-WRN (ATCC® CRL-3276™) cell line (ATCC). 50 viable crypts per well were plated in triplicate and numbers of growing, viable GI organoids were assessed at day 5 of culture. Four representative fields per well were counted at 10× Obj using the Evos FL inverted microscope and the average number from 4 fields of each of 3 replicate cultures for an individual crypt donor is reported. Images were recorded to allow quantitation of maximal cross-sectional area using Image J (Wayne Rasband, NIH). Organoids were also grown from FACS sorted single cell preparations of Lgr5+ cells obtained by a modification of the crypt isolation method used previously in order to generate a single cell suspension. Fifty Lgr5+ cells were plated per well in triplicate. Numbers of growing organoids from the entire well were counted to allow assessment of growth efficiency. Organoids were imaged and surface area calculated as above.

### RNAseq

Single cells were isolated per the MACS Miltenyi Biotec Lamina Propria dissociation kit. Cells were stained with 7AAD, CD45.2 and EpCAM and sorted per GFP expression into Lgr5⁺ and Lgr5⁻ populations. Sorting was performed on a BD FACSAria III cell sorter. TRIzol was added to single cell suspensions and cryopreserved at −80° C. RNA was subsequently extracted after a second chloroform extraction step using QIAGEN RNeasy Micro Kit. RNA libraries were prepared using the NEBnext Ultra RNA Library Prep Kit for Illumina (New England Biolabs), assessed for size and quantified using the 2100 Bioanalyzer (Agilent Technologies) and Qubit fluorometer (Thermofischer Scientific). Libraries were sequenced using high output single-end 75 cycle sequencing kits (version 2) on the Illumina Nextseq 550 platform. Sequence reads in each fastq file were trimmed for adapter sequences using Cutadapt (Martin, 2011b) (version 1.11) and aligned using STAR (Dobin et al., 2013) (version 2.5.2a) to the mm19 assembly with the gene, transcript, and exon features of Ensembl (release 75) gene model. Expression was estimated using RSEM (Li and Dewey, 2011) (version 1.2.30) and was used as input to assess differential gene expression between groups.

### Quantification and Statistical Analysis

Survival curves were plotted using Kaplan-Meier estimates and compared by log-rank analysis. The parametric nonparametric Mann-Whitney U test (two-sided) was used for comparison between two groups and 1 way ANOVA with Tukey's multiple comparison test was used for comparison between two or more groups for statistical analysis of data. Data are presented as mean±standard error of the mean (SEM). Calculations were performed using Prism 7 for windows software (GraphPad).

Sequence analysis 16S microbial sequencing. Reads were cleaned of adapter sequences using Cutadapt (Martin, 2011a) and trimmed using Trimmomatic (Bolger et al., 2014) employing a sliding window of 4 bases with an average base quality above 15, followed by hard-trimming to 250 bases with exclusion of reads less than this length. Remaining forward reads were processed following the QIIME2 workflow (https://qiime2.org) (Caporaso et al., 2010) using DADA2 (Callahan et al., 2016) to de-noise sequences. Taxonomy assignment was performed on amplicon sequence variants using BLAST (Altschul et al., 1990) against the SILVA (Quast et al., 2013) reference database version 132. Differential abundance analysis was performed on raw read counts using DESeq2 (Love et al., 2014). Counts were normalized prior to principal component analysis (PCA) and heat map visualization using cumulative sum scaling implemented within metagenomeSeq (Paulson et al., 2013). PCA was performed using the rda function within the vegan R package (Oksanen). Heat maps were generated using pheatmap (Kolde).

Intestinal cell differential gene expression was determined using the edgeR package (Robinson et al., 2010) within R v3.3.4 and significance defined as p<0.05 after Benjamini-Hochberg false discovery rate correction. Pathway analysis was performed by single sample gene set variation analysis via the GSVA package (Hanzelmann et al., 2013) using KEGG, BioCarta, Reactome and Gene Ontology (GO) pathway databases and significance defined as p<0.05 and only gene sets between 25-500 genes considered. Heat maps were generated using heatmap.2 function in gplots 3.0.1 R package. Gene set enrichment analysis (GSEA) was performed using GSEA 3.0 (Mootha et al., 2003; Subramanian et al., 2005). Canonical Pathway enrichment analysis for differentially expressed genes (log 2 Fold-change>|0.58| and adj. p-value<0.05) across PEG-IL29-treated Lgr5+ and Lgr5− samples relative to genotype-matched PBS-treated samples was done using Ingenuity Pathway Analysis (IPA) (Kramer et al., 2014). IPA function enrichment was calculated using a right-tailed Fisher exact test with a threshold of significance set at P value of 0.05. Inferences in the significant activation state (z-score>|2|) of canonical pathways, upstream regulatory transcriptional regulators, cytokines and kinases were done using IPA. Positive z-scores reflect a predicted activation state, while negative z-scores reflect the inhibition of upstream regulatory activity.

### Data And Software Availability

Both open source and commercially available software was used in this study as described in context within the methods. RNAseq data from Lgr5⁻ and Lgr5+ cells derived from PBS treated and PEG-rIL-29 treated mice has been deposited under the NCBI GEO profile accession number: GEO:pending.

### Results

### IFNλ Signaling in Recipient Tissue Determines GVHD Severity

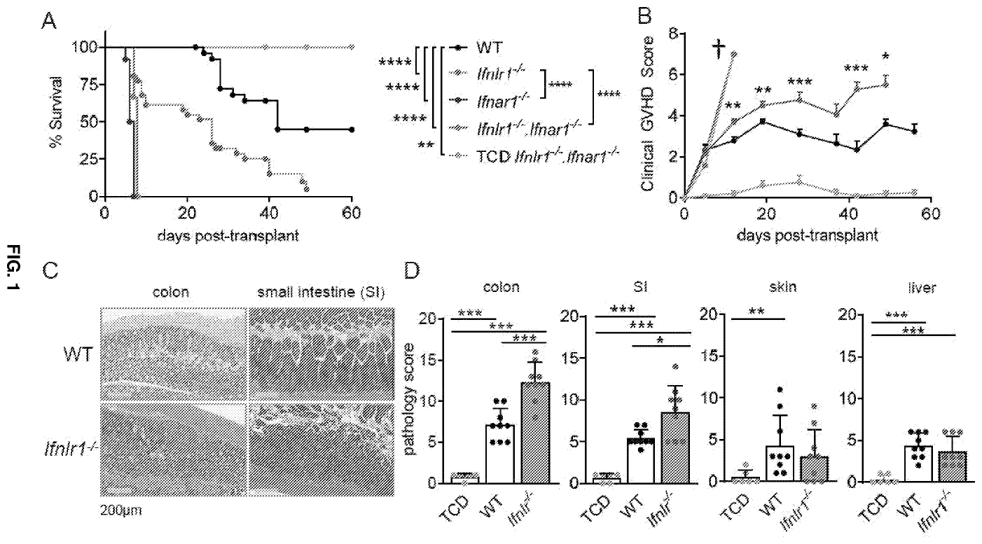

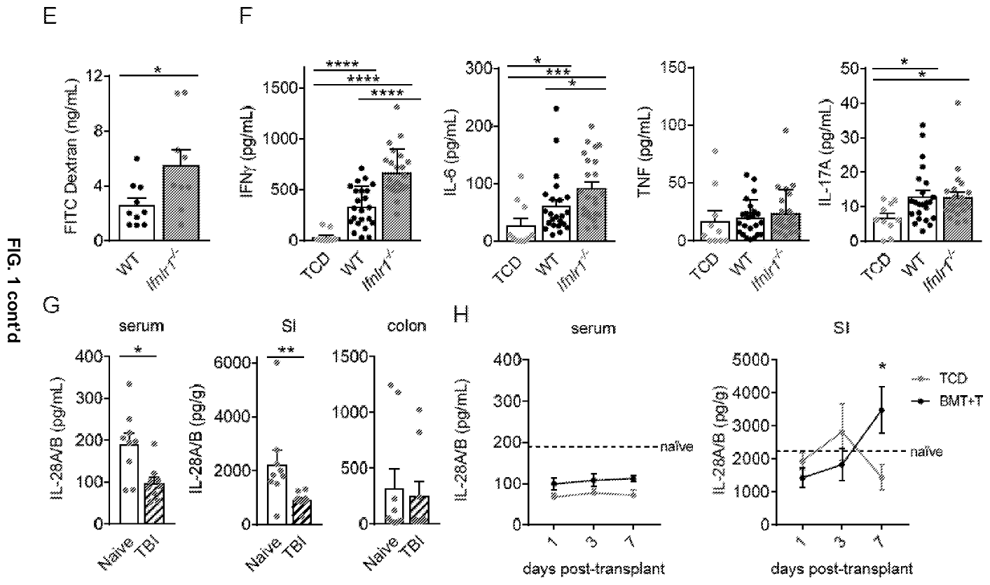

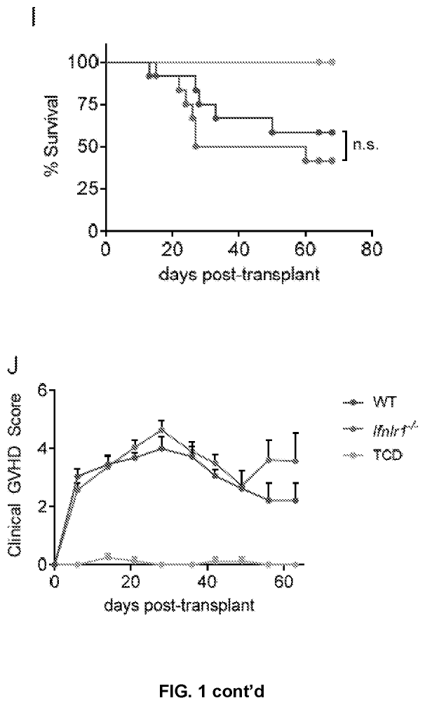

We utilized B6.Ifnlr1^{-/-} mice (henceforth referred to as Ifnlr1^{-/-}) that lack expression of the unique IFNλ receptor heterodimeric subunit Ifnlr1, rendering them unable to respond to IFNλ. These mice do not display any obvious developmental abnormalities in GI function including weight and histological appearance (FIG. 8). Ifnlr1^{-/-} mice were used as BMT recipients and outcomes were compared to B6.wild-type (WT) cohorts. Recipients of T cell depleted (TCD) grafts, that do not develop GVHD, were included as negative controls. Allogeneic BMT with full MHC-disparate BALB/c donors revealed early and more severe aGVHD, resulting in enhanced lethality of Ifnlr1^{-/-} mice (FIG. 1A). Clinical GVHD scores confirmed more severe disease in the absence of Ifnlr1 signaling (FIG. 1B). As previously published (Robb et al., 2011), recipients lacking the type I IFN receptor (Ifnar1^{-/-}) developed lethal hyper-acute GVHD within the first week after transplant and the additional absence of Ifnlr1 did not modify this (FIG. 1A). Semi-quantitative GVHD histopathology at day 7 after BMT further confirmed enhanced GVHD in the small intestine (SI) and colon in the absence of IFNλ signaling (FIG. 1C-D), indicative of a protective role within the GI tract. GVHD pathology in skin and liver was not altered in Ifnlr1^{-/-} recipients (FIG. 1D). In keeping with GI tract histopathological changes, Ifnlr1^{-/-} recipients had impaired GI tract integrity as determined by assays of barrier function (FIG. 1E). We also observed an inflammatory signature in the sera of Ifnlr1^{-/-} recipients, with significantly increased systemic dysregulation of IFNγ and IL-6 relative to WT recipients (FIG. 1F). We noted that systemic levels of IL-28 fell after total body irradiation (TBI) and this was reflected by reduced levels within small intestine (FIG. 1G). While systemic levels of IL-28 remained low after BMT, they increased in the small intestine during GVHD (FIG. 1H). In contrast to the important regulatory effect of IFNλ signaling in recipient tissue, signaling in donor cells did not modify GVHD survival (FIG. 1I) or clinical scores (FIG. 1J). We confirmed that GVL function against primary MLL-AF9 oncogene driven acute myeloid leukemia (AML) was intact in the absence of IFNλ signaling in donor cells (FIG. 9A). Similar capacity to control a Philadelphia chromosome driven blast crisis AML was also seen when WT and Ifnlr1^{-/-} donor grafts were used in this model (FIG. 9B). We thus subsequently focused on the protective mechanisms of IFNλ signaling in recipient tissues.

### Enhanced GVHD in Ifnlr1^{-/-} Recipients is Dependent on Signaling in Both Hematopoietic and Non-hematopoietic Cells

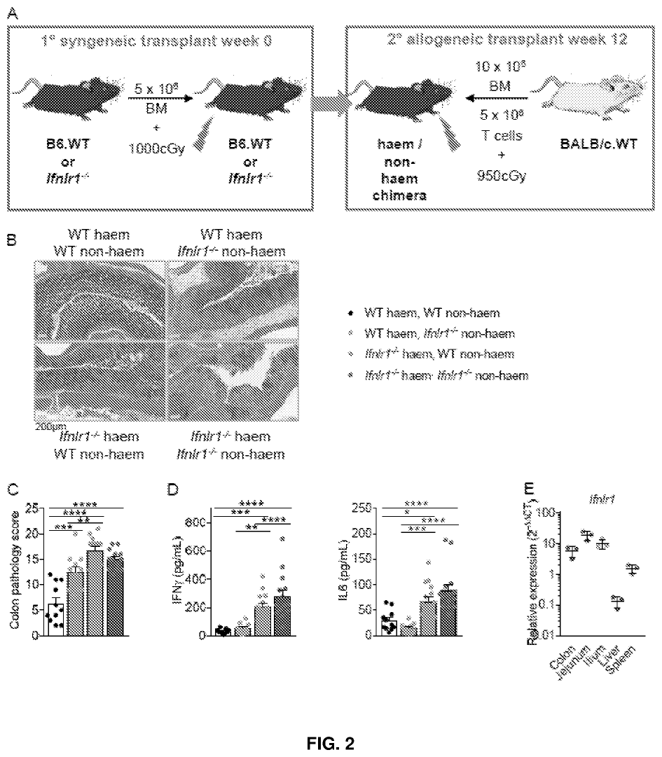

To define whether the protective role of Ifnlr1 signaling is within cells that initiate GVHD (i.e. hematopoietic) or within GVHD targets (non-hematopoietic tissue), we generated bone marrow chimeras lacking Ifnlr1 in hematopoietic and/or non-hematopoietic tissues as previously described (Burman et al., 2007; Robb et al., 2011) (FIG. 2A). Three months later, these chimeric mice were used as secondary transplant recipients and GVHD outcomes were observed. Transplant recipients lacking Ifnlr1 signaling in either the hematopoietic or non-hematopoietic compartments or both had enhanced GVHD histopathology in the colon (FIG. 2B-C). Serum levels of IFNγ and IL-6 were elevated in recipients lacking Ifnlr1 signaling in hematopoietic tissues only (FIG. 2D). These data suggest multiple and tissue specific mechanisms by which IFNλ signaling protects from GVHD. Since donor T cells are the source of excess systemic IFNγ production during GVHD (Burman et al., 2007), the protective effect of Ifnlr1 signaling in non-hematopoietic tissue in isolation appeared to be independent of effects on donor immunity and thus likely a consequence of signaling within the GI tract directly. To confirm this possibility, we analyzed Ifnlr1 gene expression in the GI tract and noted high levels of Ifnlr1 mRNA in naïve GI mucosal tissues (FIG. 2E) as has been observed by others (Mordstein et al., 2010; Sommereyns et al., 2008). Indeed, mRNA expression of Ifnlr1 in colon and SI was over 5-10-fold higher than that in liver and spleen. Thus, direct IFNλ-mediated protection within the GI epithelial compartment is highly plausible.

### Ifnlr1-signaling in Recipient NK Cells is Responsible for the Protection from GVHD Mediated by Hematopoietic Cells

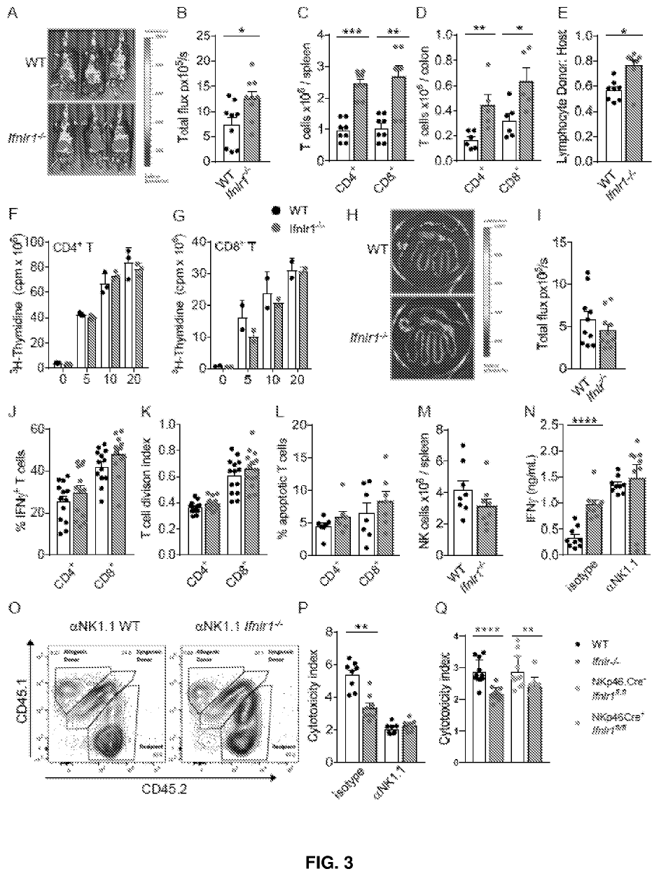

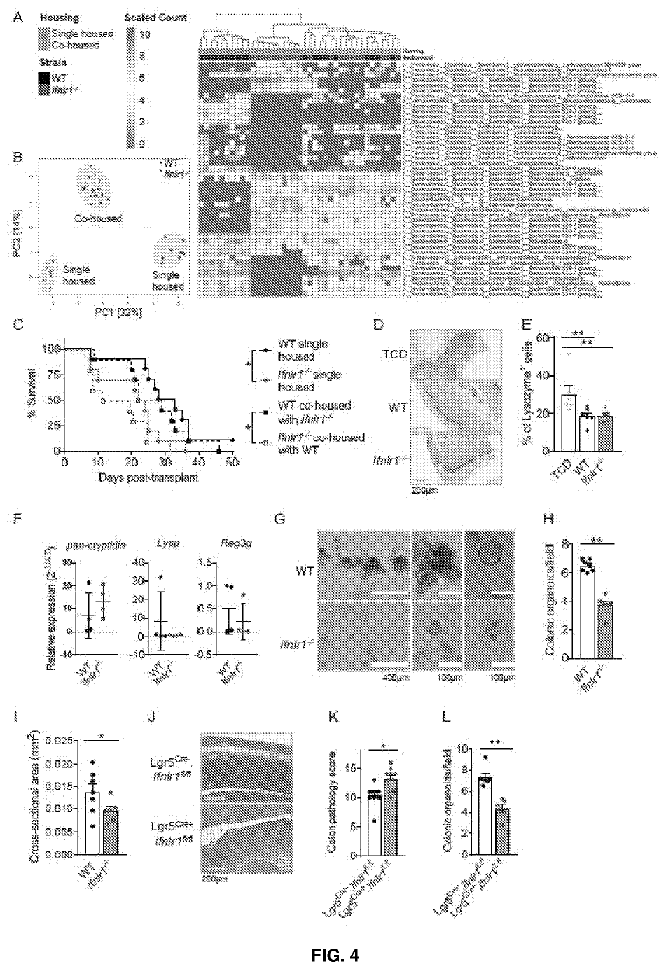

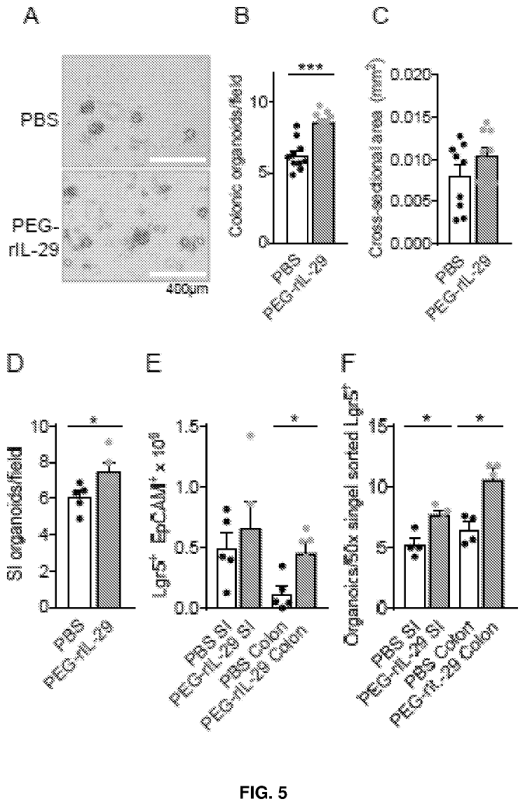

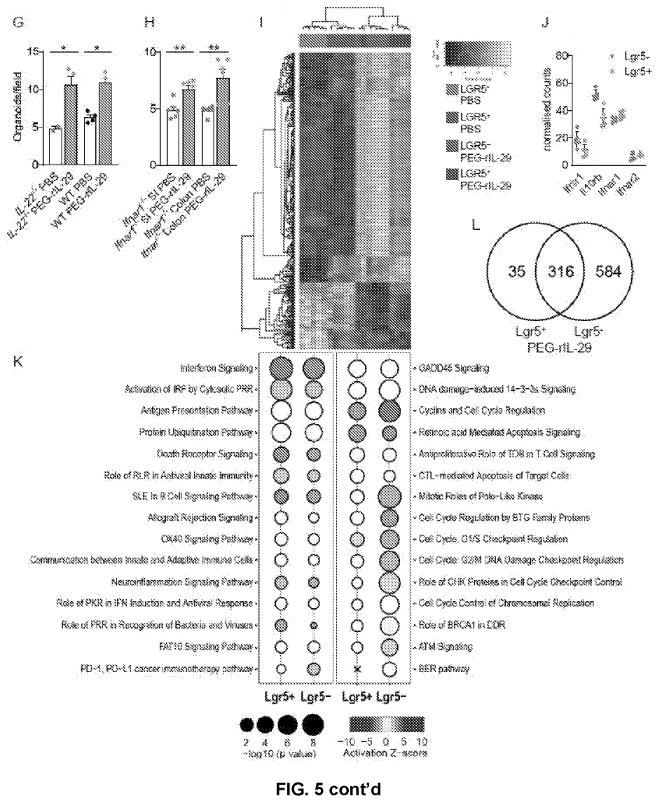

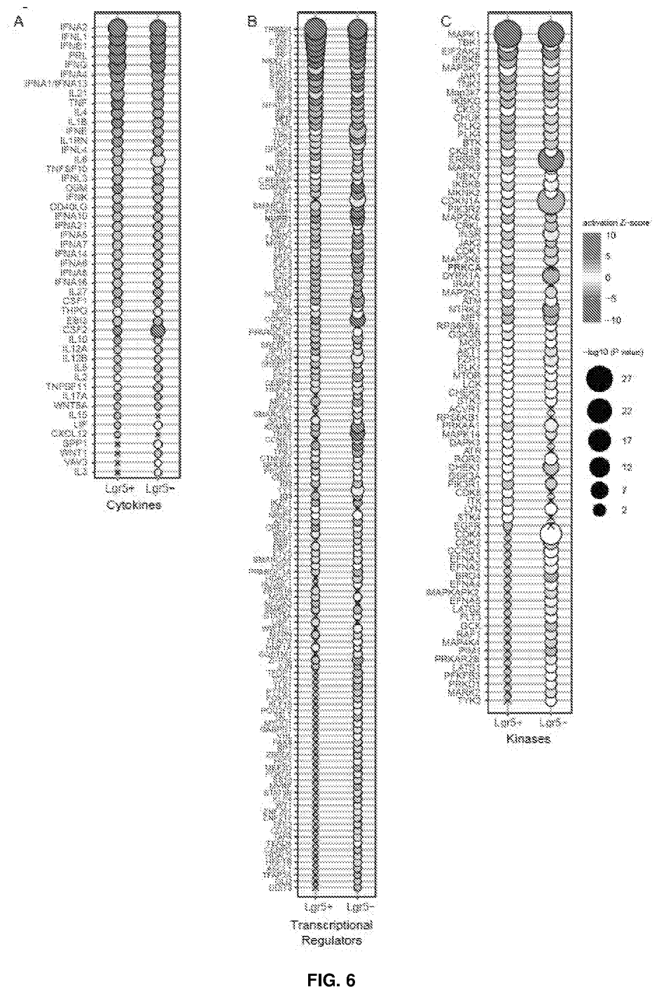

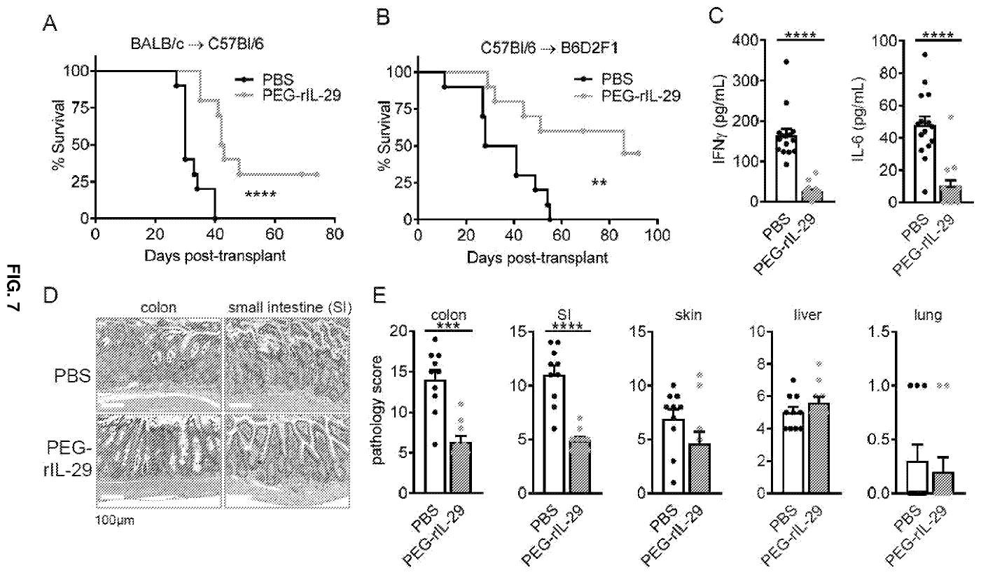

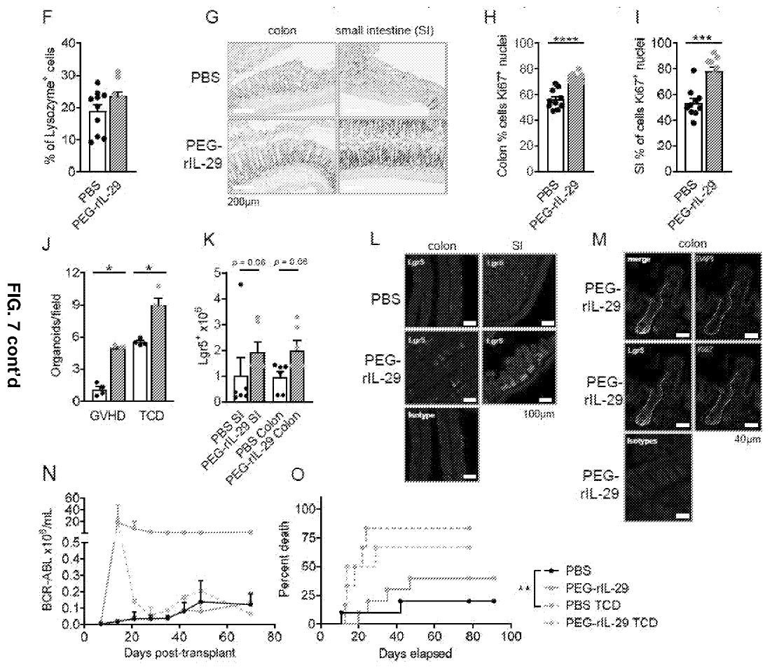

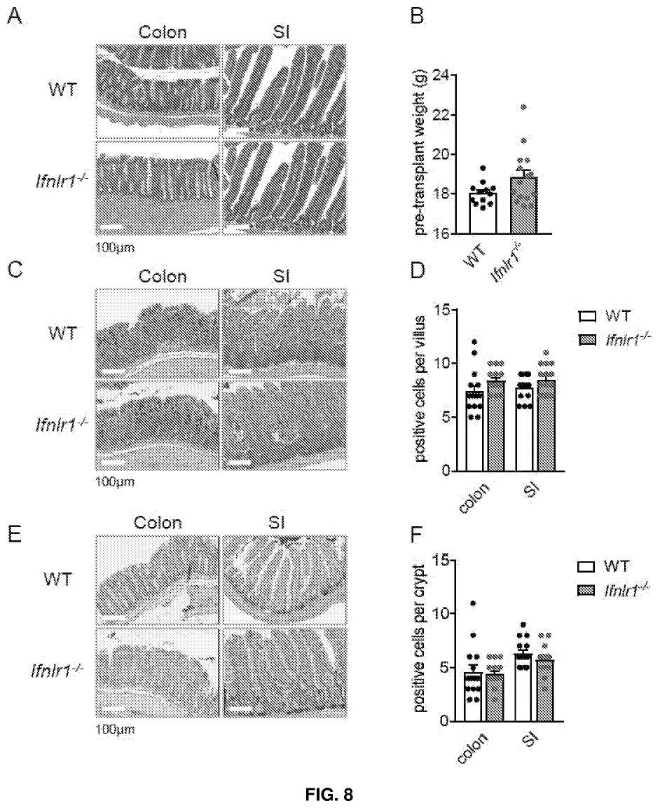

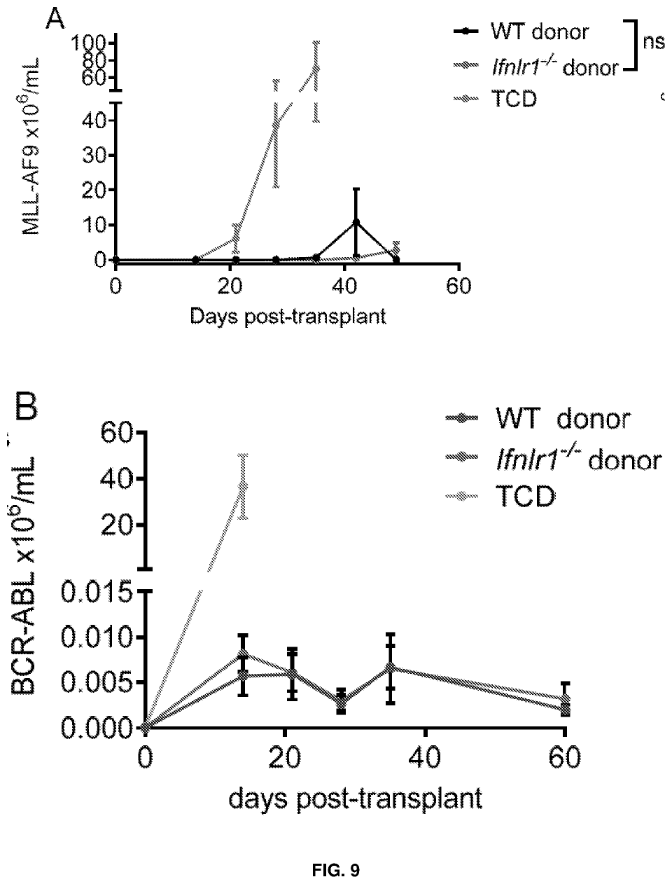

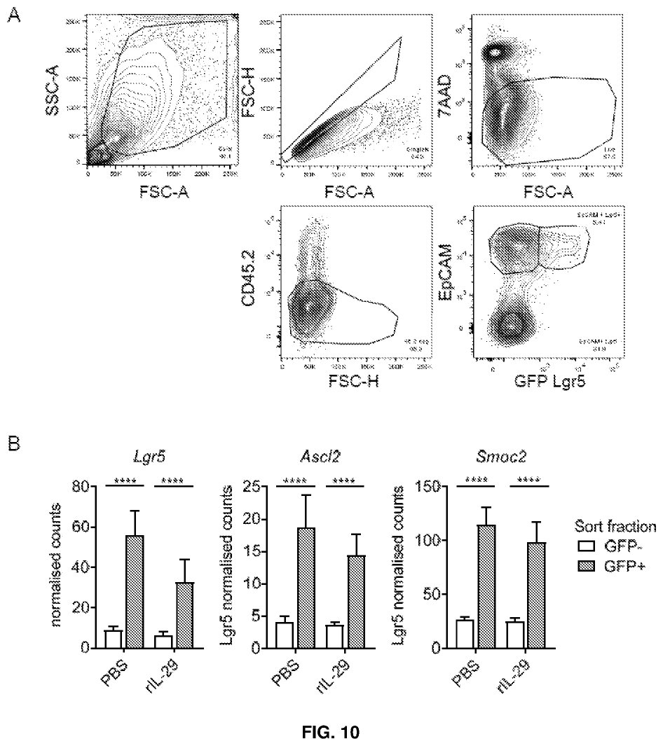

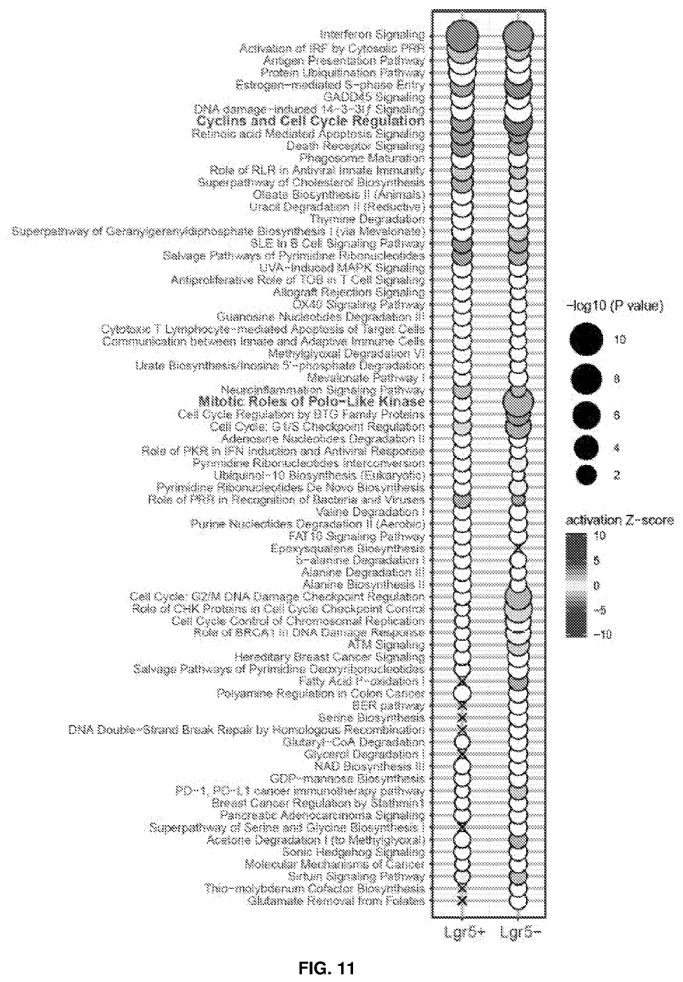

We next sought to determine the IFNλ-responsive hematopoietic cell that attenuates aGVHD and the inflammatory cytokine phenotype observed. Initial characterization of immune cell lineages revealed no differences in either myeloid or lymphoid subset frequencies and absolute numbers in Ifnlr1^{-/-} mice (data not shown). Interrogation of the donor T cell response after BMT revealed enhanced expansion of CD4+ and CD8+ cells in Ifnlr1^{-/-} relative to WT recipients. This was evident by measurement of total T cell expansion via quantification of donor T cell-derived bioluminescence in vivo (FIG. 3A-B) and early donor T cell expansion in both the spleen (FIG. 3C) and colon (FIG. 3D) at day 4 post-transplantation. Within this expanding T cell compartment a greater proportion of donor-derived T cells was seen in Ifnlr1^{-/-} recipients (FIG. 3E), indicating accelerated donor T cell engraftment.

To determine whether APC function was enhanced by IFNλ, we assessed T cell responses to WT or Ifnlr1^{-/-} dendritic cells (DC) or macrophages in mixed lymphocyte cultures. No difference was seen between CD4⁺ and CD8⁺ T cell expansion when stimulated by WT or Ifnlr1^{-/-} APC (FIG. 3F-G for DC, data for macrophages not shown). DC function was also assessed in vivo using a previously described system (Koyama et al., 2015) in which alloantigen specific CD4⁺ T cell responses were quantified in response to WT or Ifnlr1^{-/-} donor DC. Again, WT and Ifnlr1^{-/-} donor DC invoked identical alloantigen-specific T cell responses (FIG. 3H-I).

Assessment of T cell function demonstrated no increase in the proportion of donor CD4⁺ or CD8⁺ T cells that were making IFNγ (FIG. 3J), confirming that the increase in IFNγ seen in sera of Ifnlr1^{-/-} recipients was a consequence of enhanced T cell expansion. We next examined donor splenic T cell proliferation and apoptosis in vivo at day 4 after BMT and again noted equivalence in Ifnlr1^{-/-} and WT recipients (FIG. 3K-L), strengthening the proposition that early enhanced donor T cell expansion likely reflected the differences in donor T cell engraftment early after BMT (FIG. 3E). Since early engraftment is recognized to be dependent on recipient NK cell function (Westerhuis, 2005), and effects of IFNλ in NK cells have been described in tumor models (Souza-Fonseca-Guimaraes et al., 2015) and in models of CMV infection (Gimeno Brias et al., 2018), we proceeded to analysis of recipient NK cells in our system. Since numbers of NK cells were demonstrated to be equivalent in WT and Ifnlr1^{-/-} animals prior to transplantation by ourselves (FIG. 3M) and others (Souza-Fonseca-Guimaraes et al., 2015), we evaluated recipient NK cell function peri-transplant using an in vivo cytotoxicity assay (Hamby et al., 2007; Westerhuis, 2005). In this system, the depletion of recipient NK cells with anti-NK1.1 antibody results in accelerated donor T cell engraftment and enhanced systemic IFNγ dysregulation in WT recipients (FIG. 3N). In contrast, this was not noted in Ifnlr1^{-/-} recipients and indeed recipient NK depletion resulted in equivalent IFNγ production in WT and Ifnlr1^{-/-} recipients (FIG. 3N). We next quantified the killing of donor cells by measuring the relative proportion of MHC-disparate allogeneic or MHC-matched syngeneic targets in the spleen within 48 hours of transplant in the presence or absence of recipient NK cells (FIG. 3O-P). When recipient NK cells were deleted by NK1.1 antibody, the enhanced anti-donor responses in WT relative to Ifnlr1^{-/-} recipients was lost. To confirm that this effect was intrinsic to Ifnlr1^{-/-} NK cells, quantification of killing of donor cells was repeated in a conditional deletion system using NKp46^{Cre}.Ifnlr1^{fl/fl} mice. Here loss of IFNλ receptor responses in NK cells in Cre-positive recipients recapitulated the impaired anti-donor responses seen in Ifnlr1^{-/-} recipients and was significantly reduced in comparison to Cre-negative counterparts (FIG. 3Q). These data identify Ifnlr1 signaling in recipient NK cells as a primary determinant of the rate of early donor engraftment after BMT and supports that this pathway is responsible for the enhanced GVHD within Ifnlr1^{-/-} recipients that is mediated by hematopoietic cells.

### Ifnlr1-signaling in Lgr5⁺ Intestinal Stem Cells is Responsible for the Protection from GVHD Mediated by Non-hematopoietic Cells

Given the importance of IFNλ in mucosal immunity and the increasing recognition of the GI tract microbiome in determining transplant outcomes (Teshima et al., 2016; Varelias et al., 2017; Zeiser et al., 2016), we evaluated the microbiota in WT vs. Ifnlr1^{-/-} mice by 16S ribosomal sequencing. Principal component analysis of the microbiota in the two trains was significantly different at baseline but coalesced after 4 weeks of co-housing (FIG. 4A-B). This period of co-housing has been demonstrated to allow equilibration of microbiota between two discrepant in-bred mouse strains (Varelias et al., 2017). At a family level, Bacterioidales family members S24-7 (Candidatus homeothermaceae) still comprised the majority of microbial species present in both WT and Ifnlr1^{-/-} mice (FIG. 4A). The most significantly differentially represented sequences were for Helicobacteraceae family members, present at up to 5% in the Ifnlr1^{-/-} separately housed strains and virtually absent in WT fecal samples (log 2 fold change 11.0353 and p=1.022⁻⁴¹). Given the observed differences in microbial composition at baseline and their convergence after co-housing we undertook BMT in separately and co-housed mice to determine if demonstrated changes in fecal microbiota were important determinants of outcome. No difference was noted in post-transplant survival when WT recipients were housed separately or with Ifnlr1^{-/-} cage controls or conversely in Ifnlr1^{-/-} recipients housed separately or with WT controls (FIG. 4C). Thus the altered composition of the microbiome in Ifnlr1^{-/-} mice was not responsible for their enhanced GVHD.

Considering alteration in microbial defenses as a factor determining post-transplant outcomes in Ifnlr1^{-/-} mice, we also evaluated Paneth cells. Paneth cells synthesize antimicrobial peptides, have been implicated in the maintenance of the GI stem cell niche (Sato et al., 2011) and are reduced in number during aGVHD (Eriguchi et al., 2012; Levine et al., 2013). We observed equivalent numbers of Paneth cells after BMT in WT and Ifnlr1^{-/-} recipients, suggesting they are unlikely to be involved in the protective effect of IFNλ within the GI tract (FIG. 4D-E). We also confirmed equivalent expression of anti-microbial peptides by Paneth cells in WT and Ifnlr1^{-/-} mice by quantitative PCR for RNA expression of Cryptidins, Lysozyme P and regenerating islet-derived protein 3 gamma (FIG. 4F). In the absence of a protective effect of IFNλ conferred via the microbiota or Paneth cells, we proceeded to evaluate a potential role within the intestinal epithelial compartment. GI organoids can be grown from stem cells present in crypt preparations when cultured within a supporting basement membrane preparation and provided with appropriate growth factors (Dedhia et al., 2016; Miyoshi and Stappenbeck, 2013; NatMethods, 2018; VanDussen et al., 2015). They are an attractive in vitro option to study GI biology as they recapitulate multiple elements of the GI mucosa and allow manipulation and isolation in real time. We isolated crypts, containing stem cells, from the colon of naïve Ifnlr1^{-/-} and WT mice and evaluated primary growth after 5 days of culture. A significantly greater number of growing organoids of a greater size were isolated from WT compared to Ifnlr1^{-/-} mice (FIG. 4 G-I), suggesting that Ifnlr1-signalling is important in the maintenance and proliferative capacity of the GI epithelia, including the stem cell compartment from which growing organoids are derived. To confirm that loss of Ifnlr 1 signaling in the Lgr5 positive stem cell compartment was relevant to GVHD phenotype seen in Inflr1^{-/-} recipients, we generated Lgr5-EGFP-IRES^{creERT2}.Ifnlr1^{fl.fl} (Lgr5^{Cre}.Ifnlr1^{fl.fl}) mice. Tamoxifen induced conditional deletion of Ifnlr1 in the stem cell compartment in Cre positive mice resulted in more severe aGVHD in the GI tract at day seven post transplantation when compared to Cre negative mice (FIG. 4J-K). We also generated organoids from both Cre positive and Cre negative crypt donors and saw a significant reduction in the number of organoids generated from Cre positive crypt donors (FIG. 4L). Together these data indicate that Ifnlr1 signaling responses in the stem cell compartment are important mediators of GI aGVHD pathology.

### IFNλ Treatment Produces a Proliferative Phenotype in GI Stem Cells

Given the protective role for Ifnlr1 signaling in prevention of early acute GI GVHD seen in our transplantation models, we sought to test the effect of IFNλ on GI epithelial growth. An Ifnlr1 ligand, PEGylated recombinant IL-29 (PEG-rIL-29), has been developed and tested in phase I-III clinical trials in humans as adjunctive therapy to antiviral agents for the treatment of Hepatitis C virus (Muir et al., 2014; Muir et al., 2010; Nelson et al., 2017). It is known to be cross-reactive with the murine Ifnlr1 (Souza-Fonseca-Guimaraes et al., 2015) and has an estimated in vivo half-life of 50-80 hours (Muir et al., 2010). Initial ability of PEG-rIL-29 to support the GI stem cell compartment was assessed through generation of colon organoids from WT mice receiving either PEG-rIL-29 or PBS for three days prior to crypt harvest. Increased numbers of colon organoids were generated in the primary growth phase from mice receiving treatment with PEG-rIL-29 compared to those receiving PBS (FIG. 5A-B), however a consistent increase in size was not seen (FIG. 5C). Similar, albeit less dramatic, effects were seen in organoids derived from the small intestine (FIG. 5D). Growth of organoids reflects fitness of the stem cell compartment (Sato and Clevers, 2013; Yui et al., 2012) and these observations led us to specifically evaluate stem cells within our treatment system. Lgr5 positivity identifies GI epithelial stem cells (Barker et al., 2007). We utilized Lgr5-EGFP-IRES^{creERT2} reporter mice to isolate GI stem cells from mice receiving treatment with PBS or PEG-rIL-29. Whilst PEG-rIL-29 did not produce a robust increase in numbers of Lgr5⁺ stem cells within the small intestine, there were significantly greater numbers in the colon (FIG. 5E). To evaluate the functional capacity of PEG-rIL-29 treated Lgr5⁺ stem cells we flow sorted single cell Lgr5⁺ preparations from mice treated with PEG-rIL-29 or PBS for three days before crypt harvest and evaluated organoid growth. PEG-rIL-29 treated Lgr5⁺ stem cells were more efficient in generating organoids (FIG. 5F) than those from control treated mice. To confirm that PEG-rIL-29 produces specific effects within the stem cell compartment we performed RNA sequencing (RNAseq) transcriptional analysis of purified live CD45.2 negative, EpCAM positive and Lgr5 positive and negative fractions of flow sorted cells from the digested colon of PBS or PEG-rIL-29 treated mice (FIG. 10A-B). Whilst major differences were observed in gene expression between epithelial cell and stem cell fractions (4385 genes differentially expressed between Lgr5⁻ and Lgr5⁺ cells derived from PBS treated mice), strikingly distinct transcriptional signatures were observed in Lgr5⁺ and Lgr5⁻ compartments after PEG-rIL-29 treatment, with differences apparent in Lgr5⁺ versus Lgr5⁻ populations (FIG. 5I). The top 25 differentially regulated genes in both the Lgr5⁺ and Lgr5⁻ compartments are listed in Table 1 and 2. Expression of both Ifnlr1 and its heterodimeric partner IL10rb was demonstrated in both Lgr5⁺ and Lgr5⁻ compartments (FIG. 5J), in addition to the expected expression of type I Interferon receptor components Ifnar1 and Ifnar2. Using canonical pathway enrichment analysis for the differentially expressed genes across PEG-rIL-29-treated Lgr5⁺ and Lgr5⁻ samples relative to genotype-matched PBS-treated samples using Ingenuity Pathway Analysis (IPA) (Kramer et al., 2014) expression of IFN-related genes were observed across both cell types (FIG. 5K) however not all genes were shared and some were uniquely upregulated in the Lgr5⁺ compartment (FIG. 5L). This was supported through predictive upstream regulator analyses which identified IFN cytokines (FIG. 6A) and Interferon regulatory factors (FIG. 6B) as regulators of the PEG-rIL-29 induced transcriptional responses. However, PEG-rIL-29 Lgr5⁻ cells induced the expression of genes involved in cellular proliferation (FIG. 5K, left). Upstream regulatory analysis focusing on potential regulatory cytokines identified several known regulators of cellular proliferation and cell cycle control (FIG. 6C). This transcriptomic control of cell cycle compliments our functional observations in organoid culture systems (FIG. 5A-B). PEG-rIL-29 also regulated various transcripts associated with various cellular metabolic processes (FIG. 11). Lastly, amongst the regulatory kinases (FIG. 6C) several MAPK were enriched in PEG-rIL-29 Lgr5− cells. Additionally, we also identified (FIG. 6B, highlighted blue) enrichment of NUPR, SPI, ID2, and ID3 in PEG-rIL-29 Lgr5− cells which we also found to be enriched only in response to type III IFN treatment (Forero et al., 2019). Since IL-22 is known to be an important cytokine fostering intestinal organoid growth (size rather than number) and protection from GVHD mediated damage in the GI tract (Hanash et al., 2012) we evaluated the efficacy of PEG-rIL-29 treatment in IL-22^{-/-} and WT mice. PEG-rIL-29 enhanced organoid growth to an equivalent extent in both WT and IL-22^{-/-} strains (FIG. 5G), demonstrating that the effect of PEG-rIL-29 is independent of IL-22. Similarly, others have suggested the maintenance of GI epithelial barrier integrity can be influenced by type I IFNs (Fischer et al., 2017). We demonstrated preservation of the growth advantage in PEG-rIL-29 treated colonic and small intestinal organoids independent of the presence of the type I IFN receptor in Ifnar1^{-/-} crypt donors (FIG. 5L). Importantly, both lambda and type I IFN receptors were expressed in Lgr5 positive and negative intestinal epithelial cells (FIG. 5H). Altogether, these data demonstrate that PEG-rIL-29 treatment enhances the growth capacity of intestinal stem cells whilst producing marked changes in RNA transcriptional profiles. Critically, these effects do not depend on the presence of IL-22 or type I IFN receptors.

### IFNλ Treatment Protects from GVHD Within the GI Tract

To test the capacity of IFNλ to prevent GVHD in vivo, recipient mice were treated with PEG-rIL-29 from day −2 to day 0 prior to transplantation. We tested both the BALB/c→B6 and B6→B6D2F1 systems, the latter to confirm results in the absence of T cell-mediated graft rejection. In both models, recipients receiving PEG-rIL-29 had prolonged survival (FIG. 7A-B) relative to those receiving PBS alone. Systemic inflammatory cytokine dysregulation (FIG. 7C), and GVHD histopathology in the GI tract was also significantly attenuated in B6 recipients of BALB/c grafts (FIG. 7D-E). No significant effects were seen in the skin, liver or lung. In addition, no alteration was seen in the numbers of Paneth cells present in the small intestine in recipients treated with PBS or PEG-rIL-29 (FIG. 7F).

Given the observed proliferative effects in intestinal stem cells seen in our in vitro experiments, we examined the proliferation of epithelia within the GI tract after BMT. A significantly greater proportion of both colon and small intestinal epithelial cells, inclusive of the crypt base site of stem cell localization, were Ki67 positive, consistent with the ability of pre-transplant PEG-rIL-29 administration to drive epithelial proliferation after BMT (FIG. 7G-I). To examine stem cell fitness after BMT, we quantified colonic organoid growth from crypt isolates obtained 7 days after BMT from mice with or without GVHD. As expected, active GVHD resulted in a marked defect in intestinal organoid growth and a beneficial effect of PEG-rIL-29 was clearly demonstrated both in recipients with or without GVHD (FIG. 7J), consistent with previous demonstrated effects in the intestinal stem cell compartment in the presence of GVHD induced by PEG-rIL-29. We then sought to determine numbers of stem cells present post-transplant with PBS or PEG-rIL-29 treatment. Quantitation by flow after digestion of the GI tract suggested a trend to increased numbers of stem cells after PEG-rIL-29 treatment (FIG. 7K). Examination of histopathological specimens using fluorescence immunohistochemistry demonstrated an increase in Lgr5⁺ stem cell numbers after treatment with PEG-rIL-29 (FIG. 7L). Dual immuno-fluorescent labelling confirmed expression of Ki67 in Lgr5⁺ cells within crypts, consistent with the increase in Ki67 staining shown broadly in GI mucosa (FIG. 7G). Together, this data functionally validate the proliferative pathways upregulated in the RNAseq analysis of PEG-rIL-29 treated Lgr5+ cells.

Finally, we sought to exclude detrimental effects of PEG-rIL-29 treatment on GVL. PEG-rIL-29 treatment of allograft recipients of GFP expressing BCR-ABL nup98HoxA9 AML did not influence leukemia growth or death due to leukemia relative to controls receiving PBS (FIG. 6N-O) indicating GVL effects mediated by donor T cells were preserved after PEG-rIL-29 treatment.

TABLE 1
RNA sequencing from sort purified single colonic epithelial cells.

| Symbol | Gene Description | logFC | P Value | FDR |
|---|---|---|---|---|
| Lgals9 | lectin, galactose binding, soluble 9 | 3.73 | 2.70E−15 | 9.73E−12 |
| Ddx58 | DEAD (Asp-Glu-Ala-Asp) box polypeptide 58 | 3.10 | 2.38E−15 | 9.73E−12 |
| Xaf1 | XIAP associated factor 1 | 4.82 | 1.36E−15 | 9.73E−12 |
| Adar | adenosine deaminase, RNA-specific | 2.19 | 6.45E−15 | 1.43E−11 |
| Rnf213 | ring finger protein 213 | 3.98 | 6.63E−15 | 1.43E−11 |
| Stat1 | signal transducer and activator of transcription 1 | 3.51 | 8.27E−15 | 1.49E−11 |
| Ifi44 | interferon-induced protein 44 | 7.55 | 1.09E−14 | 1.69E−11 |
| Irf9 | interferon regulatory factor 9 | 2.52 | 1.97E−14 | 2.66E−11 |
| Dhx58 | DEXH (Asp-Glu-X-His) box polypeptide 58 | 4.70 | 2.87E−14 | 3.11E−11 |
| Ogfr | opioid growth factor receptor | 2.01 | 2.84E−14 | 3.11E−11 |
| Parp9 | poly (ADP-ribose) polymerase family, member 9 | 2.80 | 3.47E−14 | 3.41E−11 |
| Parp14 | poly (ADP-ribose) polymerase family, member 14 | 3.10 | 4.28E−14 | 3.85E−11 |
| Trim30a | tripartite motif-containing 30A | 4.96 | 5.52E−14 | 4.26E−11 |
| Lgals3bp | lectin, galactoside-binding, soluble, 3 binding protein | 3.11 | 5.32E−14 | 4.26E−11 |
| Rtp4 | receptor transporter protein 4 | 5.11 | 6.51E−14 | 4.69E−11 |
| Gbp6 | guanylate binding protein 6 | 4.28 | 8.13E−14 | 5.50E−11 |
| Mitd1 | MIT, microtubule interacting and transport, domain | 2.06 | 1.09E−13 | 6.94E−11 |
| | containing 1 | | | |
| Tap1 | transporter 1, ATP-binding cassette, sub-family B | 3.57 | 1.41E−13 | 8.48E−11 |
| | (MDR/TAP) | | | |
| Ifi35 | interferon-induced protein 35 | 2.76 | 1.63E−13 | 9.25E−11 |
| Ly6e | lymphocyte antigen 6 complex, locus E | 2.79 | 2.41E−13 | 1.24E−10 |
| Stat2 | signal transducer and activator of transcription 2 | 3.65 | 2.31E−13 | 1.24E−10 |
| Hsh2d | haematopoietic SH2 domain containing | 2.93 | 2.89E−13 | 1.42E−10 |
| Gbp3 | guanylate binding protein 3 | 5.99 | 3.49E−13 | 1.64E−10 |
| H2-Q10 | histocompatibility 2, Q region locus 10 | 2.63 | 4.96E−13 | 2.23E−10 |
| Irgm1 | immunity-related GTPase family M member 1 | 3.56 | 5.70E−13 | 2.47E−10 |

Top 25 differentially expressed genes in ISC treated in vivo with PEG-rIL-29. RNA sequencing from sort purified single colonic stem cells (LGR5+) derived from either rIL-29 or PBS treated mice (n=5 mice per group). Log FC=the log 2 transformed fold change obtained from edgeR analyses. FDR (false discovery rate)=the Benjamini-Hochberg (FDR) adjusted P value obtained from edgeR analyses.

TABLE 2
RNA sequencing from sort purified single colonic stem cells

| Symbol | Gene Description | logFC | P Value | FDR |
|---|---|---|---|---|
| Xaf1 | XIAP associated factor 1 | 4.61 | 3.26E−15 | 3.52E−11 |
| Ifi44 | interferon-induced protein 44 | 7.55 | 1.37E−14 | 7.40E−11 |
| Irf9 | interferon regulatory factor 9 | 2.45 | 3.21E−14 | 9.90E−11 |
| Dhx58 | DEXH (Asp-Glu-X-His) box polypeptide 58 | 4.60 | 4.46E−14 | 9.90E−11 |
| Trim30a | tripartite motif-containing 30A | 5.02 | 5.49E−14 | 9.90E−11 |
| Rtp4 | receptor transporter protein 4 | 5.25 | 5.46E−14 | 9.90E−11 |
| Ddx58 | DEAD (Asp-Glu-Ala-Asp) box polypeptide 58 | 2.49 | 8.50E−14 | 1.31E−10 |
| Lgals9 | lectin, galactose binding, soluble 9 | 2.92 | 1.25E−13 | 1.32E−10 |
| Parp9 | poly (ADP-ribose) polymerase family, member 9 | 2.58 | 1.35E−13 | 1.32E−10 |
| Stat1 | signal transducer and activator of transcription 1 | 2.90 | 1.59E−13 | 1.32E−10 |
| Adar | adenosine deaminase, RNA-specific | 1.82 | 1.47E−13 | 1.32E−10 |
| Parp14 | poly (ADP-ribose) polymerase family, member 14 | 2.88 | 1.43E−13 | 1.32E−10 |
| Rnf213 | ring finger protein 213 | 3.29 | 1.22E−13 | 1.32E−10 |
| Gbp3 | guanylate binding protein 3 | 5.95 | 5.21E−13 | 4.02E−10 |
| Gbp6 | guanylate binding protein 6 | 3.74 | 6.00E−13 | 4.32E−10 |
| Sp100 | nuclear antigen Sp100 | 4.50 | 9.37E−13 | 6.02E−10 |
| Trim34a | tripartite motif-containing 34A | 2.92 | 9.46E−13 | 6.02E−10 |
| Lgals3bp | lectin, galactoside-binding, soluble, 3 binding protein | 2.58 | 1.01E−12 | 6.07E−10 |
| Mx2 | myxovirus (influenza virus) resistance 2 | 6.15 | 1.55E−12 | 7.96E−10 |
| Mitd1 | MIT, microtubule interacting and transport, domain | 1.77 | 1.47E−12 | 7.96E−10 |
| | containing 1 | | | |
| Usp18 | ubiquitin specific peptidase 18 | 6.99 | 1.54E−12 | 7.96E−10 |
| Ogfr | opioid growth factor receptor | 1.58 | 1.75E−12 | 8.22E−10 |
| Apol9a | apolipoprotein L 9a | 5.99 | 3.49E−13 | 1.64E−10 |
| Stat2 | signal transducer and activator of transcription 2 | 2.63 | 4.96E−13 | 2.23E−10 |
| Ifi27l2b | interferon, alpha-inducible protein 27 like 2B | 3.56 | 5.70E−13 | 2.47E−10 |

Top 25 differentially expressed genes in intestinal epithelial cells treated in vivo with PEG-rIL-29. RNA sequencing from sort purified single colonic epithelial cells (LGR5−) derived from either rIL-29 or PBS treated mice (n=5 mice per group). Log FC=the log 2 transformed fold change obtained from edgeR analyses. FDR (false discovery rate)=the Benjamini-Hochberg (FDR) adjusted P value obtained from edgeR analyses.

### Discussion

Here we demonstrate that IFNλ plays a dual role in preventing acute GI GVHD following murine allotransplantation through both hematopoietic and non-hematopoietic effects in recipient tissues. In the hematopoietic compartment we observed significantly impaired NK cell function in the absence of Ifnlr1 signaling, resulting in uncontrolled donor T cell expansion and subsequent recipient tissue damage. In concert with this, we found IFNλ responses in the non-hematopoietic compartment are vital for the maintenance of intestinal epithelial barrier function and regeneration after transplant. Critically, we found these protective effects can be successfully exploited therapeutically, such that PEG-rIL-29 administration prior to BMT enhanced intestinal stem cell function after transplant and attenuated aGVHD in the GI tract.

IFNλ is a key effector cytokine of mucosal immunity, with most data to date showing a unique and non-redundant role in immune defense at mucosal barriers (Baldridge et al., 2015; Ferguson et al., 2019; Lin et al., 2016; Mordstein et al., 2010; Pott et al., 2011; Selvakumar et al., 2017). This is contextually important in understanding the role we have demonstrated for IFNλ in the pathophysiology of aGVHD, an immunopathology with a predilection for damage at sites including the GI tract. Whilst responses to IFNλ are described in immune cell populations including NK cells, DC and neutrophils (Blazek et al., 2015; Broggi et al., 2017; Koltsida et al., 2011; Souza-Fonseca-Guimaraes et al., 2015; Zanoni et al., 2017), it is clear that IFNλ receptors are predominantly expressed on tissues of epithelial origin (Pott and Stockinger, 2017). This contributes to the tissue-specific activity of type III IFNs in relation to innate anti-viral effects (Baldridge et al., 2015; Galani et al., 2017; Hernandez et al., 2015; Lin et al., 2016; Pott et al., 2011; Rocha-Pereira et al., 2018). Thus, GI tract and respiratory tissues appear to mediate direct IFNλ-dependent anti-viral responses first, followed by the induction of additional innate immunological responses at high levels of viral replication or in response to alternate TLR ligand binding and preservation of GI barrier function (Odendall et al., 2017b; Ramos et al., 2019). IFNλ also induces a specific transcriptional profile after receptor ligand binding (Forero et al., 2019). In our model, we noted IFNλ-dependent recipient NK responses controlled the rate of engraftment and systemic inflammatory cytokine generation. Enhanced engraftment of donor T cells compounds conditioning-induced local tissue damage in the GI tract, linking the hematopoietic and non-hematopoietic effects of IFNλ in aGVHD, similar to that in models of viral infection (Ye et al., 2019). Our data support a model where IFNλ is important locally at epithelial sites, and in influencing systemic immune responses specifically through NK and GI stem cell compartments. These dual roles inform the role IFNλ has in specific anti-viral immunity, for example hepatitis C virus, where it is recognized to increase both the rate of spontaneous viral remission and viral clearance in the context of anti-viral treatment for HCV (Aka et al., 2014; Akkarathamrongsin et al., 2014; Ge et al., 2009; Marcello et al., 2006; Robek et al., 2005).

Our data support a specific protective effect within GI epithelia independent of immune-driven systemic inflammation. In the absence of robust and effective immunological measures to separate GVHD and GVL, protecting key tissue targets has been suggested as an effective means by which to attain this goal (Wu and Reddy, 2017). IFNλ treatment was able to directly improve proliferation and regenerative capacity of Lgr5⁺ stem cells. The GI tract however is increasingly recognized as a crossroads of immunity with the balance of local pathogen control exerted by resident immune cell populations, and immune responses within mucosal cell populations also influenced by the luminal microbial contents and the immunomodulatory products they produce (Jenq et al., 2012; Taur et al., 2014). As an immunopathology, GVHD (and transplantation) causes disruption to the integrity of the epithelial barrier, and preservation of this barrier is critical to the prevention of GVHD lethality (Koyama et al., 2015; Koyama and Hill, 2016). IFNλ appears to be beneficial in providing protection in this setting through multiple mechanisms in our models. Lack of Ifnlr1 does not result in pathological dysbiosis, nor apparent reduction in innate antimicrobial defenses mediated by Paneth cells. Ifnlr 1 mediates maintenance of GI barrier integrity and RNAseq suggests potential for direct immune-shielding in addition to the proliferative effects seen functionally in the epithelial compartment. In-vivo PEG-rIL-29 treatment of mice resulted in the regulation by Lgr5⁺ stem cells of pathways of antigen presentation and T cell mediated immunity (FIG. 5H). Amongst the top 25 differentially regulated genes between PBS and PEG-rIL-29 treated stem cells is Lgals9 or galectin 9, a ligand for the T cell inhibitory receptor Tim-3 (log fold change −2.916, p=4.36⁻⁵⁷ for PBS treated vs PEG-rIL-29) that has been shown to reduce aGVHD lethality following BMT in galectin-9 transgenic hosts (Veenstra et al., 2012), suggesting mechanisms protecting the GI epithelia from T cell mediated damage are induced by treatment, in addition to the effects on proliferation.

Our findings are in contrast with a recent study that did not identify significant IFNλ-mediated effects on mortality after BMT (Fischer et al., 2019). Unfortunately, the absence of specific data from this prior study surrounding clinical signs of GVHD, tissue pathology, as well as a range of other methodologies reported here, make these findings difficult to compare. Importantly, there were key differences between the studies with regard to transplant model systems, therapeutic products and treatment strategies that influence GVHD kinetics, GVHD severity and treatment exposure, which are highly likely account for this disparity.

The ability to shield the GI tract from GVHD damage has been a holy grail in transplantation for some time (Hill and Ferrara, 2000) but has yet to come to clinical fruition. IL-22 secretion from innate lymphoid cells has recently been shown to protect the Lgr5⁺ intestinal stem cell compartment from aGVHD (Hanash et al., 2012; Lindemans et al., 2015) including through secretion of antimicrobial peptides (Ferrara et al., 2011; Zhao et al., 2018). However, IL-22 has also been shown to mediate cutaneous GVHD (Gartlan et al., 2018) and so treatment with this cytokine may have undesired effects. In contrast, treatment with IFNλ does not appear to cause pathology within the hematopoietic compartment, specifically there does not appear to be effects on antigen presenting cells or alterations to T cell function that are likely to impair GVL or pathogen-specific immunity. In contrast, improving NK cell responses could arguably improve this component of the GVL response, particularly in patients receiving T cell depleted grafts where this pathway is maximally active and conditioning intensity is often very high, causing significant GI tract toxicity (Bunting et al., 2017).

Manipulation of cytokine signaling has a generated significant interest as a method to interfere with immune-mediated pathologies, where GVHD represents an extreme example (Hanash et al., 2012; Kennedy et al., 2014; Kleist et al., 2011; Koreth et al., 2011). To date cytoprotection within the GI tract has been explored in relation to a number of cytokines including IL-11 and keratinocyte growth factor (Hill et al., 1998; Krijanov ski et al., 1999; Panoskaltsis-Mortari et al., 1998; Teshima et al., 1999). The latter has shown clinical activity in relation to limiting the severity of mucositis in chemoradiotherapy-conditioned allotransplant patients but this has not translated to the attenuation of aGVHD (Blazar et al., 2006; Hill and Ferrara, 2000). More recently, IL-22 has shown to be capable of preserving Paneth cell numbers and the stem cell niche to attenuate aGVHD in the GI tract (Hanash et al., 2012; Lindemans et al., 2015). Importantly, the effects mediated by IFNλ are independent of IL-22. Most recently, recipient-derived IL-17A has been shown to be important in preventing dysbiosis and maintaining intestinal barrier function after BMT, an effect at least partially mediated by MALT cells (Varelias et al., 2018; Varelias et al., 2017). Thus a number of cytokines in the IFN and IL-17/IL-22 family appear important and clinically tractable to enhance intestinal barrier function (Ferguson et al., 2019; Odendall et al., 2017a). The availability of clinical grade IFNλ with an established safety profile makes our findings rapidly translatable and testable in the clinic.

There is clear potential for the use of IFNλ outside of BMT, particularly in other immunopathologies that cause specific damage to GI epithelia such as inflammatory bowel disease (Pott and Stockinger, 2017; Vlachiotis and Andreakos, 2019). Regeneration of epithelial components in ulcerative colitis would rationally limit morbidity and the link to disease severity and luminal microbial components has also been made in Crohn's disease (Gevers et al., 2014; Manichanh et al., 2006). Reduction of morbidity associated with therapeutic radiation of fields encompassing the GI tract is an additional logical application for the effects seen in our experiments. Similarly, specific GI tract protection from damage as a consequence of intensive chemotherapy regimens, such as those during induction therapy for acute myeloid leukemia is also testable. Our data provides a sound basis for the therapeutic exploitation of IFNλ mediated protection of the GI epithelia in allogeneic BMT that avoids off target effects on leukemia and pathogen-specific immunity.

All computer programs, algorithms, patent, sequences of the accession numbers and scientific literature referred to herein is incorporated herein by reference.

### REFERENCES

The Editors, Aka, P. V., Kuniholm, M. H., Pfeiffer, R. M., Wang, A. S., Tang, W., Chen, S., Astemborski, J., Plankey, M., Villacres, M. C., Peters, M. G., et al. (2014). Association of the IFNL4-ΔG Allele With Impaired Spontaneous Clearance of Hepatitis C Virus. J. Infect. Dis. 209, 350-354.

Akkarathamrongsin, S., Thong, V. D., Payungporn, S., Poovorawan, K., Prapunwattana, P., Poovorawan, Y., and Tangkijvanich, P. (2014). IFNL3 (IL28B) and IFNL4 polymorphisms are associated with treatment response in Thai patients infected with HCV genotype 1, but not with genotypes 3 and 6. J. Med. Virol. 86, 1482-1490.

Altschul, S. F., Gish, W., Miller, W., Myers, E. W., and Lipman, D. J. (1990). Basic local alignment search tool. Journal of Molecular Biology 215, 403-410.

Baldridge, M. T., Nice, T. J., McCune, B. T., Yokoyama, C. C., Kambal, A., Wheadon, M., Diamond, M. S., Ivanova, Y., Artyomov, M., and Virgin, H. W. (2015). Commensal microbes and interferon-determine persistence of enteric murine norovirus infection. Science 347, 266-269.

Barker, N., van Es, J. H., Kuipers, J., Kujala, P., van den Born, M., Cozijnsen, M., Haegebarth, A., Korving, J., Begthel, H., Peters, P. J., and Clevers, H. (2007). Identification of stem cells in small intestine and colon by marker gene Lgr5. Nature 449, 1003-1007.

Bhushal, S., Wolfsmuller, M., Selvakumar, T. A., Kemper, L., Wirth, D., Hornef, M. W., Hauser, H., and Koster, M. (2017). Cell Polarization and Epigenetic Status Shape the Heterogeneous Response to Type III Interferons in Intestinal Epithelial Cells. Front Immunol 8, 671.

Blazar, B. R., Weisdorf, D. J., Defor, T., Goldman, A., Braun, T., Silver, S., and Ferrara, J. L. (2006). Phase ½ randomized, placebo-control trial of palifermin to prevent graft-versus-host disease (GVHD) after allogeneic hematopoietic stem cell transplantation (HSCT). Blood 108, 3216-3222.

Blazek, K., Eames, H. L., Weiss, M., Byrne, A. J., Perocheau, D., Pease, J. E., Doyle, S., McCann, F., Williams, R. O., and Udalova, I. A. (2015). IFN- resolves inflammation via suppression of neutrophil infiltration and IL-1 production. Journal of Experimental Medicine 212, 845-853.

Bolger, A. M., Lohse, M., and Usadel, B. (2014). Trimmomatic: a flexible trimmer for Illumina sequence data. Bioinformatics 30, 2114-2120.

Broggi, A., Tan, Y., Granucci, F., and Zanoni, I. (2017). IFN-lambda suppresses intestinal inflammation by non-translational regulation of neutrophil function. Nat Immunol 18, 1084-1093.

Bunting, M. D., Varelias, A., Souza-Fonseca-Guimaraes, F., Schuster, I. S., Lineburg, K. E., Kuns, R. D., Fleming, P., Locke, K. R., Huntington, N. D., Blazar, B. R., et al. (2017). GVHD prevents NK-cell-dependent leukemia and virus-specific innate immunity. Blood 129, 630-642.

Burman, A. C., Banovic, T., Kuns, R. D., Clouston, A. D., Stanley, A. C., Morris, E. S., Rowe, V., Bofinger, H., Skoczylas, R., Raffelt, N., et al. (2007). IFN differentially controls the development of idiopathic pneumonia syndrome and GVHD of the gastrointestinal tract. Blood 110, 1064-1072.

Callahan, B. J., McMurdie, P. J., Rosen, M. J., Han, A. W., Johnson, A. J., and Holmes, S. P. (2016). DADA2: High-resolution sample inference from Illumina amplicon data. Nat Methods 13, 581-583.

Caporaso, J. G., Kuczynski, J., Stombaugh, J., Bittinger, K., Bushman, F. D., Costello, E. K., Fierer, N., Pena, A. G., Goodrich, J. K., Gordon, J. I., et al. (2010). QIIME allows analysis of high-throughput community sequencing data. Nat Methods 7, 335-336.

Casazza, R. L., and Lazear, H. M. (2019). Why Is IFN-lambda Less Inflammatory? One IRF Decides. Immunity 51, 415-417.

Cooke, K. R., Kobzik, L., Martin, T. R., Brewer, J., Delmonte, J., Jr., Crawford, J. M., and Ferrara, J. L. (1996). An experimental model of idiopathic pneumonia syndrome after bone marrow transplantation: I. The roles of minor H antigens and endotoxin. Blood 88, 3230-3239.

Dedhia, P. H., Bertaux-Skeirik, N., Zavros, Y., and Spence, J. R. (2016). Organoid Models of Human Gastrointestinal Development and? Disease. Gastroenterology 150, 1098-1112.

Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics (Oxford, England) 29, 15-21.

Dumoutier, L., Lejeune, D., Hor, S., Fickenscher, H., and Renauld, J.-C. (2003). Cloning of a new type II cytokine receptor activating signal transducer and activator of transcription (STAT)1, STAT2 and STAT3. Biochemical Journal 370, 391.

Eriguchi, Y., Takashima, S., Oka, H., Shimoji, S., Nakamura, K., Uryu, H., Shimoda, S., Iwasaki, H., Shimono, N., Ayabe, T., et al. (2012). Graft-versus-host disease disrupts intestinal microbial ecology by inhibiting Paneth cell production of -defensins. Blood 120, 223-231.

Ferguson, S. H., Foster, D. M., Sherry, B., Magness, S. T., Nielsen, D. M., and Gookin, J. L. (2019). Interferon-lambda3 Promotes Epithelial Defense and Barrier Function Against Cryptosporidium parvum Infection. Cell Mol Gastroenterol Hepatol 8, 1-20.

Ferrara, J. L., Harris, A. C., Greenson, J. K., Braun, T. M., Holler, E., Teshima, T., Levine, J. E., Choi, S. W., Huber, E., Landfried, K., et al. (2011). Regenerating islet-derived 3-alpha is a biomarker of gastrointestinal graft-versus-host disease. Blood 118, 6702-6708.

Fischer, J. C., Bscheider, M., Eisenkolb, G., Lin, C.-C., Wintges, A., Otten, V., Lindemans, C. A., Heidegger, S., Rudelius, M., Monette, S., et al. (2017). RIG-I/MAVS and STING signaling promote gut integrity during irradiation- and immune-mediated tissue injury. Science Translational Medicine 9, eaag2513.

Fischer, J. C., Lin, C. C., Heidegger, S., Wintges, A., Schlapschy, M., Beudert, M., Combs, S. E., Bassermann, F., Skerra, A., Haas, T., and Poeck, H. (2019). Regeneration After Radiation- and Immune-Mediated Tissue Injury Is Not Enhanced by Type III Interferon Signaling. Int J Radiat Oncol Biol Phys 103, 970-976.

Forero, A., Ozarkar, S., Li, H., Lee, C. H., Hemann, E. A., Nadjsombati, M. S., Hendricks, M. R., So, L., Green, R., Roy, C. N., et al. (2019). Differential Activation of the Transcription Factor IRF1 Underlies the Distinct Immune Responses Elicited by Type I and Type III Interferons. Immunity 51, 451-464 e456.

Galani, I. E., Triantafyllia, V., Eleminiadou, E.-E., Koltsida, O., Stavropoulos, A., Manioudaki, M., Thanos, D., Doyle, S. E., Kotenko, S. V., Thanopoulou, K., and Andreakos, E. (2017). Interferon-λ Mediates Non-redundant Front-Line Antiviral Protection against Influenza Virus Infection without Compromising Host Fitness. Immunity 46, 875-890.e876.

Gartlan, K. H., Bommiasamy, H., Paz, K., Wilkinson, A. N., Owen, M., Reichenbach, D. K., Banovic, T., Wehner, K., Buchanan, F., Varelias, A., et al. (2018). A critical role for donor-derived IL-22 in cutaneous chronic GVHD. Am J Transplant 18, 810-820.

Ge, D., Fellay, J., Thompson, A. J., Simon, J. S., Shianna, K. V., Urban, T. J., Heinzen, E. L., Qiu, P., Bertelsen, A. H., Muir, A. J., et al. (2009). Genetic variation in IL28B predicts hepatitis C treatment-induced viral clearance. Nature 461, 399-401.

Gevers, D., Kugathasan, S., Denson, L. A., Vazquez-Baeza, Y., Van Treuren, W., Ren, B., Schwager, E., Knights, D., Song, S. J., Yassour, M., et al. (2014). The treatment-naive microbiome in new-onset Crohn's disease. Cell Host Microbe 15, 382-392.

Gimeno Brias, S., Marsden, M., Forbester, J., Clement, M., Brandt, C., Harcourt, K., Kane, L., Chapman, L., Clare, S., and Humphreys, I. R. (2018). Interferon lambda is required for interferon gamma-expressing NK cell responses but does not afford antiviral protection during acute and persistent murine cytomegalovirus infection. PLoS One 13, e0197596.

Hamby, K., Trexler, A., Pearson, T. C., Larsen, C. P., Rigby, M. R., and Kean, L. S. (2007). NK Cells Rapidly Reject Allogeneic Bone Marrow in the Spleen Through a Perforin- and Ly49D-Dependent, but NKG2D-Independent Mechanism. American Journal of Transplantation 7, 1884-1896.

Hamming, O. J., Terczyliska-Dyla, E., Vieyres, G., Dijkman, R., Jørgensen, S. E., Akhtar, H., Siupka, P., Pietschmann, T., Thiel, V., and Hartmann, R. (2013). Interferon lambda 4 signals via the IFNλ receptor to regulate antiviral activity against HCV and coronaviruses: Recombinant IFNλ4 is antiviral. The EMBO Journal 32, 3055-3065.

Hanash, Alan M., Dudakov, Jarrod A., Hua, G., O'Connor, Margaret H., Young, Lauren F., Singer, Natalie V., West, Mallory L., Jenq, Robert R., Holland, Amanda M., Kappel, Lucy W., et al. (2012). Interleukin-22 Protects Intestinal Stem Cells from Immune-Mediated Tissue Damage and Regulates Sensitivity to Graft versus Host Disease. Immunity 37, 339-350.

Hanzelmann, S., Castelo, R., and Guinney, J. (2013). GSVA: gene set variation analysis for microarray and RNA-seq data. BMC Bioinformatics 14, 7.

Hernández, P. P., Mahlakzõiv, T., Yang, I., Schwierzeck, V., Nguyen, N., Guendel, F., Gronke, K., Ryffel, B., Hölscher, C., Dumoutier, L., et al. (2015). Interferon-λ and interleukin 22 act synergistically for the induction of interferon-stimulated genes and control of rotavirus infection. Nature Immunology 16, 698-707.

Hill, G. R., Cooke, K. R., Teshima, T., Crawford, J. M., Keith, J. C., Jr., Brinson, Y. S., Bungard, D., and Ferrara, J. L. (1998). Interleukin-11 promotes T cell polarization and prevents acute graft-versus-host disease after allogeneic bone marrow transplantation. J Clin Invest 102, 115-123.

Hill, G. R., Crawford, J. M., Cooke, K. R., Brinson, Y. S., Pan, L., and Ferrara, J. L. (1997). Total body irradiation and acute graft-versus-host disease: the role of gastrointestinal damage and inflammatory cytokines. Blood 90, 3204-3213.

Hill, G. R., and Ferrara, J. L. (2000). The primacy of the gastrointestinal tract as a target organ of acute graft-versus-host disease: rationale for the use of cytokine shields in allogeneic bone marrow transplantation. Blood 95, 2754-2759.

Jenq, R. R., Ubeda, C., Taur, Y., Menezes, C. C., Khanin, R., Dudakov, J. A., Liu, C., West, M. L., Singer, N. V., Equinda, M. J., et al. (2012). Regulation of intestinal inflammation by microbiota following allogeneic bone marrow transplantation. Journal of Experimental Medicine 209, 903-911.

Kennedy, G. A., Varelias, A., Vuckovic, S., Le Texier, L., Gartlan, K. H., Zhang, P., Thomas, G., Anderson, L., Boyle, G., Cloonan, N., et al. (2014). Addition of interleukin-6 inhibition with tocilizumab to standard graft-versus-host disease prophylaxis after allogeneic stem-cell transplantation: a phase ½ trial. The Lancet Oncology 15, 1451-1459.

Kleist, B., Xu, L., Li, G., and Kersten, C. (2011). Expression of the adult intestinal stem cell marker Lgr5 in the metastatic cascade of colorectal cancer. Int J Clin Exp Pathol 4, 327-335.

Kolde, R. (2015). pheatmap: Pretty Heatmaps.

Koltsida, O., Hausding, M., Stavropoulos, A., Koch, S., Tzelepis, G., Übel, C., Kotenko, S. V., Sideras, P., Lehr, H. A., Tepe, M., et al. (2011). IL-28A (IFN-λ2) modulates lung DC function to promote Th1 immune skewing and suppress allergic airway disease: IL-28 promotes Th1 skewing and suppresses asthma. EMBO Molecular Medicine 3, 348-361.

Koreth, J., Matsuoka, K.-i., Kim, H. T., McDonough, S. M., Bindra, B., Alyea, E. P., Armand, P., Cutler, C., Ho, V. T., Treister, N. S., et al. (2011). Interleukin-2 and Regulatory T Cells in Graft-versus-Host Disease. New England Journal of Medicine 365, 2055-2066.

Kotenko, S. V., Gallagher, G., Baurin, V. V., Lewis-Antes, A., Shen, M., Shah, N. K., Langer, J. A., Sheikh, F., Dickensheets, H., and Donnelly, R. P. (2002). IFN-λs mediate antiviral protection through a distinct class II cytokine receptor complex. Nature Immunology 4, 69-77.

Koyama, M., Cheong, M., Markey, K. A., Gartlan, K. H., Kuns, R. D., Locke, K. R., Lineburg, K. E., Teal, B. E., Leveque-El Mouttie, L., Bunting, M. D., et al. (2015). Donor colonic CD103+ dendritic cells determine the severity of acute graft-versus-host disease. J Exp Med 212, 1303-1321.

Koyama, M., and Hill, G. R. (2016). Alloantigen presentation and graft-versus-host disease: fuel for the fire. Blood 127, 2963-2970.

Kramer, A., Green, J., Pollard, J., Jr., and Tugendreich, S. (2014). Causal analysis approaches in Ingenuity Pathway Analysis. Bioinformatics 30, 523-530.

Krijanovski, O. I., Hill, G. R., Cooke, K. R., Teshima, T., Crawford, J. M., Brinson, Y. S., and Ferrara, J. L. (1999). Keratinocyte growth factor separates graft-versus-leukemia effects from graft-versus-host disease. Blood 94, 825-831.

Kunin, V., Engelbrektson, A., Ochman, H., and Hugenholtz, P. (2010). Wrinkles in the rare biosphere: pyrosequencing errors can lead to artificial inflation of diversity estimates. Environ Microbiol 12, 118-123.

Lasfar, A. (2006). Characterization of the Mouse IFN- Ligand-Receptor System: IFN-s Exhibit Antitumor Activity against B16 Melanoma. Cancer Research 66, 4468-4477.

Levine, J. E., Huber, E., Hammer, S. T. G., Harris, A. C., Greenson, J. K., Braun, T. M., Ferrara, J. L. M., and Holler, E. (2013). Low Paneth cell numbers at onset of gastrointestinal graft-versus-host disease identify patients at high risk for nonrelapse mortality. Blood 122, 1505-1509.

Li, B., and Dewey, C. N. (2011). RSEM: accurate transcript quantification from RNA-Seq data with or without a reference genome. BMC bioinformatics 12, 323.

Lin, J.-D., Feng, N., Sen, A., Balan, M., Tseng, H.-C., McElrath, C., Smirnov, S. V., Peng, J., Yasukawa, L. L., Durbin, R. K., et al. (2016). Distinct Roles of Type I and Type III Interferons in Intestinal Immunity to Homologous and Heterologous Rotavirus Infections. PLOS Pathogens 12, e1005600.

Lindemans, C. A., Calafiore, M., Mertelsmann, A. M., O'Connor, M. H., Dudakov, J. A., Jenq, R. R., Velardi, E., Young, L. F., Smith, O. M., Lawrence, G., et al. (2015). Interleukin-22 promotes intestinal-stem-cell-mediated epithelial regeneration. Nature 528, 560-564.

Love, M. I., Huber, W., and Anders, S. (2014). Moderated estimation of fold change and dispersion for RNA-seq data with DESeq2. Genome Biol 15, 550.

Manichanh, C., Rigottier-Gois, L., Bonnaud, E., Gloux, K., Pelletier, E., Frangeul, L., Nalin, R., Jarrin, C., Chardon, P., Marteau, P., et al. (2006). Reduced diversity of faecal microbiota in Crohn's disease revealed by a metagenomic approach. Gut 55, 205-211.

Marcello, T., Grakoui, A., Barba-Spaeth, G., Machlin, E. S., Kotenko, S. V., MacDonald, M. R., and Rice, C. M. (2006). Interferons alpha and lambda inhibit hepatitis C virus replication with distinct signal transduction and gene regulation kinetics. Gastroenterology 131, 1887-1898.

Martin, M. (2011a). Cutadapt removes adapter sequences from high-throughput sequencing reads. EMBnet.journal 17.

Martin, M. (2011b). Cutadapt removes adapter sequences from high-throughput sequencing reads. EMBnet.journal 17, 10-12.

Miyoshi, H., and Stappenbeck, T. S. (2013). In vitro expansion and genetic modification of gastrointestinal stem cells in spheroid culture. Nat Protoc 8, 2471-2482.

Mootha, V. K., Lindgren, C. M., Eriksson, K. F., Subramanian, A., Sihag, S., Lehar, J., Puigserver, P., Carlsson, E., Ridderstrale, M., Laurila, E., et al. (2003). PGC-1alpha-responsive genes involved in oxidative phosphorylation are coordinately downregulated in human diabetes. Nature genetics 34, 267-273.

Mordstein, M., Neugebauer, E., Ditt, V., Jessen, B., Rieger, T., Falcone, V., Sorgeloos, F., Ehl, S., Mayer, D., Kochs, G., et al. (2010). Lambda Interferon Renders Epithelial Cells of the Respiratory and Gastrointestinal Tracts Resistant to Viral Infections. Journal of Virology 84, 5670-5677.

Muir, A. J., Arora, S., Everson, G., Flisiak, R., George, J., Ghalib, R., Gordon, S. C., Gray, T., Greenbloom, S., Hassanein, T., et al. (2014). A Randomized Phase 2b Study of Peginterferon Lambda-1a for the Treatment of Chronic HCV Infection. Journal of Hepatology.

Muir, A. J., Shiffman, M. L., Zaman, A., Yoffe, B., de la Torre, A., Flamm, S., Gordon, S. C., Marotta, P., Vierling, J. M., Lopez-Talavera, J. C., et al. (2010). Phase 1b study of pegylated interferon lambda 1 with or without ribavirin in patients with chronic genotype 1 hepatitis C virus infection. Hepatology 52, 822-832.

NatMethods, E. (2018). Method of the Year 2017: Organoids. Nature Methods 15, 1-1.

Nelson, M., Rubio, R., Lazzarin, A., Romanova, S., Luetkemeyer, A., Conway, B., Molina, J. M., Xu, D., Srinivasan, S., and Portsmouth, S. (2017). Safety and Efficacy of Pegylated Interferon Lambda, Ribavirin, and Daclatasvir in HCV and HIV-Coinfected Patients. J Interferon Cytokine Res 37, 103-111.

Odendall, C., Voak, A. A., and Kagan, J. C. (2017a). Type III IFNs Are Commonly Induced by Bacteria-Sensing TLRs and Reinforce Epithelial Barriers during Infection. J Immunol 199, 3270-3279.

Odendall, C., Voak, A. A., and Kagan, J. C. (2017b). Type III IFNs Are Commonly Induced by Bacteria-Sensing TLRs and Reinforce Epithelial Barriers during Infection. The Journal of Immunology 199, 3270-3279.

Oksanen, J., F. G. Blanchet, R. Kindt, P. Legendre, P. R. Minchin, R. B. O'Hara, G. L. Simpson, P. Solymos, M. H. H. Stevens, and H. Wagner. (2015). vegan: Community Ecology Package.

Panoskaltsis-Mortari, A., Lacey, D. L., Vallera, D. A., and Blazar, B. R. (1998). Keratinocyte Growth Factor Administered Before Conditioning Ameliorates Graft-Versus-Host Disease After Allogeneic Bone Marrow Transplantation in Mice. Blood 92, 3960-3967.

Paulson, J. N., Stine, O. C., Bravo, H. C., and Pop, M. (2013). Differential abundance analysis for microbial marker-gene surveys. Nat Methods 10, 1200-1202.

Pott, J., Mahlakoiv, T., Mordstein, M., Duerr, C. U., Michiels, T., Stockinger, S., Staeheli, P., and Hornef, M. W. (2011). IFN- determines the intestinal epithelial antiviral host defense. PNAS 108, 7944-7949.

Pott, J., and Stockinger, S. (2017). Type I and III Interferon in the Gut: Tight Balance between Host Protection and Immunopathology. Front Immunol 8, 258.

Prokunina-Olsson, L., Muchmore, B., Tang, W., Pfeiffer, R. M., Park, H., Dickensheets, H., Hergott, D., Porter-Gill, P., Mumy, A., Kohaar, I., et al. (2013). A variant upstream of IFNL3 (IL28B) creating a new interferon gene IFNL4 is associated with impaired clearance of hepatitis C virus. Nature Genetics 45, 164-171.

Quast, C., Pruesse, E., Yilmaz, P., Gerken, J., Schweer, T., Yarza, P., Peplies, J., and Glockner, F. O. (2013). The SILVA ribosomal RNA gene database project: improved data processing and web-based tools. Nucleic Acids Res 41, D590-596.

Ramos, I., Smith, G., Ruf-Zamojski, F., Martinez-Romero, C., Fribourg, M., Carbajal, E. A., Hartmann, B. M., Nair, V. D., Marjanovic, N., Monteagudo, P. L., et al. (2019). Innate Immune Response to Influenza Virus at Single-Cell Resolution in Human Epithelial Cells Revealed Paracrine Induction of Interferon Lambda 1. J Virol 93.

Robb, R. J., Kreijveld, E., Kuns, R. D., Wilson, Y. A., Olver, S. D., Don, A. L. J., Raffelt, N. C., De Weerd, N. A., Lineburg, K. E., Varelias, A., et al. (2011). Type I-IFNs control GVHD and GVL responses after transplantation. Blood 118, 3399-3409.

Robek, M. D., Boyd, B. S., and Chisari, F. V. (2005). Lambda Interferon Inhibits Hepatitis B and C Virus Replication. Journal of Virology 79, 3851-3854.

Robinson, M. D., McCarthy, D. J., and Smyth, G. K. (2010). edgeR: a Bioconductor package for differential expression analysis of digital gene expression data. Bioinformatics 26, 139-140.

Rocha-Pereira, J., Jacobs, S., Noppen, S., Verbeken, E., Michiels, T., and Neyts, J. (2018). Interferon lambda (IFN-λ) efficiently blocks norovirus transmission in a mouse model. Antiviral Research 149, 7-15.

Sato, T., and Clevers, H. (2013). Growing self-organizing mini-guts from a single intestinal stem cell: mechanism and applications. Science 340, 1190-1194.

Sato, T., van Es, J. H., Snippert, H. J., Stange, D. E., Vries, R. G., van den Born, M., Barker, N., Shroyer, N. F., van de Wetering, M., and Clevers, H. (2011). Paneth cells constitute the niche for Lgr5 stem cells in intestinal crypts. Nature 469, 415-418.

Selvakumar, T. A., Bhushal, S., Kalinke, U., Wirth, D., Hauser, H., Koster, M., and Hornef, M. W. (2017). Identification of a Predominantly Interferon-lambda-Induced Transcriptional Profile in Murine Intestinal Epithelial Cells. Front Immunol 8, 1302.

Sheppard, P., Kindsvogel, W., Xu, W., Henderson, K., Schlutsmeyer, S., Whitmore, T. E., Kuestner, R., Garrigues, U., Birks, C., Roraback, J., et al. (2002). IL-28, IL-29 and their class II cytokine receptor IL-28R. Nature Immunology 4, 63-68.

Simms-Waldrip, T. R., Sunkersett, G., Coughlin, L. A., Savani, M. R., Arana, C., Kim, J., Kim, M., Zhan, X., Greenberg, D. E., Xie, Y., et al. (2017). Antibiotic-Induced Depletion of Anti-inflammatory Clostridia Is Associated with the Development of Graft-versus-Host Disease in Pediatric Stem Cell Transplantation Patients. Biology of Blood and Marrow Transplantation 23, 820-829.

Sommereyns, C., Paul, S., Staeheli, P., and Michiels, T. (2008). IFN-Lambda (IFN-λ) Is Expressed in a Tissue-Dependent Fashion and Primarily Acts on Epithelial Cells In Vivo. PLoS Pathogens 4, e1000017.

Souza-Fonseca-Guimaraes, F., Young, A., Mittal, D., Martinet, L., Bruedigam, C., Takeda, K., Andoniou, C. E., Degli-Esposti, M. A., Hill, G. R., and Smyth, M. J. (2015). NK cells require IL-28R for optimal in vivo activity. PNAS 112, E2376-E2384.

Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: A knowledge-based approach for interpreting genome-wide expression profiles. Proceedings of the National Academy of Sciences 102, 15545-15550.

Tanaka, Y., Nishida, N., Sugiyama, M., Kurosaki, M., Matsuura, K., Sakamoto, N., Nakagawa, M., Korenaga, M., Hino, K., Hige, S., et al. (2009). Genome-wide association of IL28B with response to pegylated interferon-a and ribavirin therapy for chronic hepatitis C. Nature Genetics 41, 1105-1109.

Taur, Y., Jenq, R. R., Perales, M. A., Littmann, E. R., Morjaria, S., Ling, L., No, D., Gobourne, A., Viale, A., Dahi, P. B., et al. (2014). The effects of intestinal tract bacterial diversity on mortality following allogeneic hematopoietic stem cell transplantation. Blood 124, 1174-1182.

Teshima, T., Hill, G. R., Pan, L., Brinson, Y. S., van den Brink, M. R., Cooke, K. R., and Ferrara, J. L. (1999). IL-11 separates graft-versus-leukemia effects from graft-versus-host disease after bone marrow transplantation. J Clin Invest 104, 317-325.

Teshima, T., Reddy, P., and Zeiser, R. (2016). Acute Graft-versus-Host Disease: Novel Biological Insights. Biology of Blood and Marrow Transplantation 22, 11-16.

VanDussen, K. L., Marinshaw, J. M., Shaikh, N., Miyoshi, H., Moon, C., Tarr, P. I., Ciorba, M. A., and Stappenbeck, T. S. (2015). Development of an enhanced human gastrointestinal epithelial culture system to facilitate patient-based assays. Gut 64, 911-920.

Varelias, A., Bunting, M. D., Ormerod, K. L., Koyama, M., Olver, S. D., Straube, J., Kuns, R. D., Robb, R. J., Henden, A. S., Cooper, L., et al. (2018). Recipient mucosal-associated invariant T cells control GVHD within the colon. J Clin Invest 128, 1919-1936.

Varelias, A., Ormerod, K. L., Bunting, M. D., Koyama, M., Gartlan, K. H., Kuns, R. D., Lachner, N., Locke, K. R., Lim, C. Y., Henden, A. S., et al. (2017). Acute graft-versus-host disease is regulated by an IL-17—sensitive microbiome. Blood 129, 2172-2185.

Veenstra, R. G., Taylor, P. A., Zhou, Q., Panoskaltsis-Mortari, A., Hirashima, M., Flynn, R., Liu, D., Anderson, A. C., Strom, T. B., Kuchroo, V. K., and Blazar, B. R. (2012). Contrasting acute graft-versus-host disease effects of Tim-3/galectin-9 pathway blockade dependent upon the presence of donor regulatory T cells. Blood 120, 682-690.

Vlachiotis, S., and Andreakos, E. (2019). Lambda interferons in immunity and autoimmunity. J Autoimmun 104, 102319.

Westerhuis, G. (2005). Long-term mixed chimerism after immunologic conditioning and MHC-mismatched stem-cell transplantation is dependent on NK-cell tolerance. Blood 106, 2215-2220.

Wu, S. R., and Reddy, P. (2017). Tissue tolerance: a distinct concept to control acute GVHD severity. Blood 129, 1747-1752.

Ye, L., Schnepf, D., Becker, J., Ebert, K., Tanriver, Y., Bernasconi, V., Gad, H. H., Hartmann, R., Lycke, N., and Staeheli, P. (2019). Interferon-lambda enhances adaptive mucosal immunity by boosting release of thymic stromal lymphopoietin. Nat Immunol 20, 593-601.

Yui, S., Nakamura, T., Sato, T., Nemoto, Y., Mizutani, T., Zheng, X., Ichinose, S., Nagaishi, T., Okamoto, R., Tsuchiya, K., et al. (2012). Functional engraftment of colon epithelium expanded in vitro from a single adult Lgr5(+) stem cell. Nat Med 18, 618-623.

Zanoni, I., Granucci, F., and Broggi, A. (2017). Interferon (IFN)-λ Takes the Helm: Immunomodulatory Roles of Type III IFNs. Frontiers in Immunology 8.

Zeiser, R., Socié, G., and Blazar, B. R. (2016). Pathogenesis of acute graft-versus-host disease: from intestinal microbiota alterations to donor T cell activation. British Journal of Haematology 175, 191-207.

Zhao, D., Kim, Y. H., Jeong, S., Greenson, J. K., Chaudhry, M. S., Hoepting, M., Anderson, E. R., van den Brink, M. R., Peled, J. U., Gomes, A. L., et al. (2018). Survival signal REG3alpha prevents crypt apoptosis to control acute gastrointestinal graft-versus-host disease. J Clin Invest.

1. A method of preventing or treating a gastrointestinal disease, disorder or condition in a subject, said method including the step of administering to the subject a therapeutically effective amount of an IFNλ receptor agonist to thereby prevent or treat the gastrointestinal disease, disorder or condition in the subject.

2. The method of claim 1, wherein the gastrointestinal disease, disorder or condition is at least partly characterized by gastrointestinal epithelial damage and/or loss.

3. The method of claim 1 or claim 2, wherein the gastrointestinal disease, disorder or condition is selected from the group consisting of an inflammatory intestinal disease, an infection, an autoimmune disease, immunotherapy-induced intestinal damage, an immune deficiency, a haematological malignancy, chemotherapy-induced intestinal damage, radiation-induced intestinal damage, graft versus host disease (GVHD), and any combination thereof.

4. The method of claim 3, wherein the gastrointestinal disease, disorder or condition is or comprises GVHD.

5. The method of claim 4, wherein the GVHD is or comprises acute and/or chronic GVHD.

6. The method of claim 4 or claim 5, wherein the GVHD is or comprises intestinal and/or colonic epithelial damage and/or loss.

7. The method of any one of the preceding claims, wherein the subject has been administered or is administered an immunosuppressive agent.

8. The method of any one of the preceding claims, wherein the IFNλ receptor agonist is administered to the subject prior to, simultaneously with and/or subsequent to administration of a transplant to the subject.

9. The method of claim 8, wherein the transplant is or comprises a hematopoietic stem cell transplant.

10. The method of claim 8 or claim 9, wherein the IFNλ receptor agonist does not modulate and/or preserves graft versus leukemia (GVL) and/or graft versus tumour (GVT) effects of the transplant.

11. A method of promoting survival and/or proliferation of an intestinal cell, said method including the step of contacting the intestinal cell with an IFNλ receptor agonist under conditions to promote survival and/or proliferation of the intestinal cell.

12. The method of claim 11, wherein the intestinal cell is or comprises an intestinal stem cell.

13. The method of claim 12, wherein the intestinal stem cell is positive for or expresses one or more of Lgr5, Ascl2 and Smoc2.

14. The method of any one of claims 11 to 14, wherein the method is performed in vitro or in vivo in a subject.

15. The method of claim 14, wherein the IFNλ receptor agonist is administered prior to, simultaneously with and/or subsequent to administration of a cytotoxic agent to the subject.

16. The method of claim 15, wherein the cytotoxic agent is or comprises a chemotherapeutic agent and/or a radiotherapy.

17. The method of any one of claims 14 to 16, wherein the subject has or is suffering from a gastrointestinal disease, disorder or condition.

18. The method of claim 17, wherein the gastrointestinal disease, disorder or condition is selected from the group consisting of an inflammatory intestinal disease, an infection, an autoimmune disease, immunotherapy-induced intestinal damage, an immune deficiency, a haematological malignancy, chemotherapy-induced intestinal damage, radiation-induced intestinal damage, GVHD, and any combination thereof.

19. A method of performing a transplant in a subject in need thereof, said method including the step of administering to the subject a therapeutically effective amount of an IFNλ receptor agonist prior to, simultaneously with and/or subsequent to administration of the transplant to the subject.

20. The method of claim 19, wherein the transplant is or comprises a haematopoietic stem cell transplant.

21. The method of claim 19 or 20, including the further step of administering the transplant to the subject.

22. An IFNλ receptor agonist for use in:
(a) preventing or treating a gastrointestinal disease, disorder or condition;
(b) promoting survival and/or proliferation of an intestinal cell; and/or
(c) performing a transplant;
in a subject.

23. Use of an IFNλ receptor agonist in the manufacture of a medicament for:
(a) preventing or treating a gastrointestinal disease, disorder or condition;
(b) promoting survival and/or proliferation of an intestinal cell; and/or
(c) performing a transplant;
in a subject.

24. The method of any one of claims 1 to 21, the IFNλ receptor agonist of claim 22 or the use of claim 23, wherein the IFNλ receptor agonist is selected from the group consisting of IFN-λ1 (IL-29), IFN-λ2 (IL-28A), IFN-λ3 (IL-28B), IFN-λ4, including variants and derivatives thereof and any combination thereof.

25. The method, IFNλ receptor agonist or use of claim 24, wherein the IFNλ receptor agonist is or comprises recombinant IL-29, preferably pegylated recombinant IL-29.
